(19) United States
(12) Patent Application Publication
(10) Pub. No.: US 2022/0357314 A1
(43) Pub. Date: Nov. 10, 2022

(54) Cell-based Clostridal Neurotoxin Assays

(71) Applicants: IPSEN BIOPHARM LIMITED, Wrexham (GB); INSTITUTE OF MOLECULAR AND CELL BIOLOGY, BIOMEDICAL SCIENCES INSTITUTES, Singapore (SG)

(72) Inventors: Keith FOSTER, Wrexham (GB); Matthew BEARD, Wrexham (GB); Jeremy Changyu YEO, Singapore (SG); Frederic Andre Jean BARD, Singapore (SG); Pei Ling Felicia TAY, Singapore (SG)

(21) Appl. No.: 17/278,465

(22) PCT Filed: Sep. 27, 2019
(86) PCT No.: PCT/GB2019/052734

§ 371 Date: Mar. 22, 2021

(30) Foreign Application Priority Data
Sep. 28, 2018 (GB) 1815870.9

(51) Int. Cl. G01N 33/50; C07K 14/435

(52) U.S. Cl. CPC G01N 33/5058; C07K 14/43595; G01N 2333/33; C07K 2319/60

(57) ABSTRACT

The present invention is directed to a method for identifying a gene that regulates clostridial neurotoxin activity, the method comprising: a. providing a sample of human neuronal cells expressing a polypeptide that comprises a C-terminal detectable label, wherein the polypeptide is cleavable by a clostridial neurotoxin; b. altering expression of a target gene of the cells; c. contacting the cells with the clostridial neurotoxin; d. measuring an amount of C-terminal detectable label, thereby quantifying clostridial neurotoxin activity; and e. identifying the target gene as a regulator of clostridial neurotoxin activity when the quantified clostridial neurotoxin activity is different to the quantified clostridial neurotoxin activity when expression of the target gene is unaltered; or f. identifying that the target gene is not a regulator of clostridial neurotoxin activity when the quantified clostridial neurotoxin activity is equivalent to the quantified clostridial neurotoxin activity when expression of the target gene is unaltered. Also provided are related methods for identifying an agent that regulates clostridial neurotoxin activity, as well as human neuronal cells, nucleotides, vectors, polypeptides, kits, and compositions suitable for use in the methods of the invention.

The present invention relates to cell-based clostridial neurotoxin assays.

Bacteria in the genus Clostridia produce highly potent and specific protein toxins, which can poison neurons and other cells to which they are delivered. Examples of such clostridial neurotoxins include the neurotoxins produced by C. tetani (TeNT) and by C. botulinum (BoNT) serotypes A-G, and X (see WO 2018/009903 A2), as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters.

While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

The mouse LD₅₀ assay is currently the only assay approved by the FDA for release of botulinum toxins. The assay simultaneously tests the action of all three domains of a botulinum neurotoxin (i.e. binding, translocation, and protease). In more detail, it defines the median lethal intraperitoneal dose of the toxin at a defined time-point usually 2-4 days after dosing (activity is expressed in mouse LD₅₀ units). Regrettably, however, LD₅₀ assays use large numbers of animals. Moreover, LD₅₀ units are not absolute measurements because they are not biological constants—as such they are highly dependent on the assay conditions. In particular, errors associated with this assay can be as high as 60% between different testing facilities (Sesardic et al. 2003; Biologicals 31(4):265-276).

The mouse flaccid paralysis assay, which is also known as the ‘mouse abdominal ptosis assay’, relates the activity of botulinum toxin to the degree of abdominal bulging seen after the toxin is subcutaneously injected into the left inguinocrural region of a mouse—the magnitude of the paralysis is dose-dependent. This approach has been proposed as a refinement to the mouse LD₅₀ test, because it relies on a humane endpoint. This assay is approximately 10 times more sensitive than the LD₅₀ assay, uses a sub-lethal dose of toxin and is more rapid than the LD₅₀ test as it provides results in 24 to 48 hours, compared to 72 to 96 hours for a typical LD₅₀ assay. The results from this assay show excellent agreement with the LD₅₀ values (Sesardic et al., 1996; Pharmacol Toxicol, 78(5): 283-8). Although this assay uses 20% of the animals used in the LD₅₀ assay it still necessitates the use of animals.

Assays such as the mouse/rat phrenic nerve hemi-diaphragm assay (which are based on the use of ex vivo nerve/muscle preparations) relate the activity of botulinum neurotoxin to a decrease in the amplitude of a twitch response of the preparation after it is applied to a maintenance medium. The usual endpoint of the assay is the time required before a 50% decrease in amplitude is observed. Regrettably, however, the hemi-diaphragm assay (like the LD₅₀ assay) results in the use of large numbers of animals. In addition, the assay requires highly skilled personnel trained in the use of sophisticated and expensive equipment.

All of the above assays have particular failings, notably animal welfare issues. Moreover, none of the above-mentioned assays are suitable for high throughput testing, for example for detecting genetic or chemical regulators of clostridial neurotoxin. Thus, there is a need in the art for alternative and/or improved clostridial neurotoxin assays.

The present invention overcomes one or more of the above-mentioned problems.

In one aspect the invention provides a method for identifying a gene that regulates clostridial neurotoxin activity, the method comprising:
a. providing a sample of human neuronal cells expressing a polypeptide that comprises a C-terminal detectable label, wherein the polypeptide is cleavable by a clostridial neurotoxin;
b. altering expression of a target gene of the cells;
c. contacting the cells with the clostridial neurotoxin;
d. measuring an amount of C-terminal detectable label, thereby quantifying clostridial neurotoxin activity; and
e. identifying the target gene as a regulator of clostridial neurotoxin activity when the quantified clostridial neurotoxin activity is different to the quantified clostridial neurotoxin activity when expression of the target gene is unaltered.

Alternatively, the method may comprise identifying that the target gene is not a regulator of clostridial neurotoxin activity when the quantified clostridial neurotoxin activity is equivalent to the quantified clostridial neurotoxin activity when expression of the target gene is unaltered.

In one embodiment the cells may be contacted with a clostridial neurotoxin prior to altering expression of the target gene.

In a related aspect the invention provides a method for identifying an agent that regulates clostridial neurotoxin activity, the method comprising:
a. providing a sample of human neuronal cells expressing a polypeptide that comprises a C-terminal detectable label, wherein the polypeptide is cleavable by a clostridial neurotoxin;
b. contacting the cells with the clostridial neurotoxin and an agent, wherein the contacting is sequential or simultaneous;
c. measuring an amount of C-terminal detectable label, thereby quantifying clostridial neurotoxin activity; and
d. identifying the agent as a regulator of clostridial neurotoxin activity when the quantified clostridial neurotoxin activity is different to the quantified clostridial neurotoxin activity in the absence of the agent.

Alternatively, the method may comprise identifying that the agent is not a regulator of clostridial neurotoxin activity when the quantified clostridial neurotoxin activity is equivalent to the quantified clostridial neurotoxin activity in the absence of the agent.

An agent may be identified as a negative regulator of clostridial neurotoxin activity when the quantified clostridial neurotoxin activity is less than the quantified clostridial neurotoxin activity in the absence of the agent. Alternatively, an agent may be identified as a positive regulator of clostridial neurotoxin activity when the quantified clostridial neurotoxin activity is greater than the quantified clostridial neurotoxin activity in the absence of the agent.

The present invention employs the use of human neuronal cells and is associated with a number of advantages absent in conventional cell-based assays, such as those employing the use of murine cells. The human neuronal cells allow for the use of human gene silencing libraries (e.g. human siRNA libraries) or agent libraries (e.g. human drug compound libraries). Thus, the present invention has higher predictive capabilities than conventional cell-based assays and has improved human therapeutic relevance.

Thus, in one aspect the invention provides a human neuronal cell expressing a polypeptide that is cleavable by a clostridial neurotoxin and comprises a C-terminal detectable label. The cell is preferably a stable cell line comprising a nucleotide sequence or vector of the invention.

The human neuronal cell of the invention is preferably a non-cancer cell. Preferably the human neuronal cell of the invention is an immortalized human neural progenitor cell or cell equivalent thereto (e.g. functionally equivalent thereto). For example the cell may be a ReNcell® Human Neural Progenitor (commercially available from Sigma-Aldrich) expressing a polypeptide of the invention. Advantageously, non-cancer cells are genetically closer to native human neurons as compared to cancer cell lines (e.g. neuroblastoma cells), and thus represent an improved neuronal cell model for use in the assays described herein.

The human neural progenitor cell is preferably differentiated prior to use in a method of the invention. Thus in one embodiment a human neuronal cell of the invention or for use in a method of the invention (e.g. a human neuron or precursor thereto) is derived from a human neural progenitor cell. More preferably, the human neuronal cell of the invention or for use in a method of the invention is a human neuron or a cell equivalent thereto (e.g. functionally equivalent thereto).

A polypeptide expressed by a human neuronal cell described herein may comprise both an N-terminal and C-terminal detectable label. In one embodiment the N-terminal and C-terminal detectable labels are different.

The detectable label is preferably a fluorescent label. In some embodiments a polypeptide of the invention does not comprise a further non-fluorescent label.

In one embodiment either the N-terminal or C-terminal detectable label is a red fluorescent protein (RFP). More preferably, one terminal of the protein has a RFP detectable label and the other terminal of the protein has a detectable label selected from: green fluorescent protein (GFP), cyan fluorescent protein (CFP), and yellow fluorescent protein (YFP). Advantageously, RFP is antigenically distinct from GFP, CFP, and YFP, therefore the methods of the invention allow the use of (secondary/confirmatory) immunogenic detection techniques, such as Western blotting. Polypeptides where both labels are selected from GFP, CFP and YFP are typically not suitable for use with such immunogenic detection techniques in view of antibody cross-reactivity. Preferably a polypeptide described herein comprises a N-terminal RFP detectable label and a C-terminal GFP detectable label.

It is preferred that the polypeptide of the invention does not comprise a tag for immobilisation and/or purification; as the assay is cell-based such tags are unnecessary. In one embodiment, the polypeptide of the invention may not comprise a His-tag (e.g. a poly-histidine tag, such as a 6-His tag), a FLAG-tag, a Protein A tag, a maltose binding protein tag, and/or a Myc-tag.

Therefore, in one aspect the invention provides a polypeptide that is cleavable by a clostridial neurotoxin and comprises an N-terminal RFP detectable label and a C-terminal GFP detectable label. In a related aspect the invention provides a nucleotide sequence encoding said polypeptide, as well as a vector (e.g. a plasmid) comprising a nucleotide sequence of the invention operably linked to a promoter. Any promoter suitable for expression in a human neuronal cell may be used, such as a CMV promoter.

The polypeptide comprises a substrate of a clostridial neurotoxin or a portion thereof. The substrate (or portion thereof) is suitably selected based on the clostridial neurotoxin being assayed.

In one embodiment the polypeptide comprises a substrate of a botulinum neurotoxin (or a portion thereof), such as a SNARE protein. Thus, the polypeptide may comprise a substrate (or portion thereof) of BoNT/A, BoNT/B, BoNT/C1, BoNT/D, BoNT/E, BoNT/F, BoNT/G or BoNT/X. Preferably, the polypeptide comprises a substrate (or portion thereof) of BoNT/A.

A polypeptide of the invention may comprise synaptosomal-associated protein of 25 kDa (SNAP-25), a synaptobrevin/vesicle-associated membrane protein (VAMP, e.g. VAMP1, VAMP2, VAMP3, VAMP4 or VAMP5), syntaxin (e.g. syntaxin 1, syntaxin 2 or syntaxin 3), Ykt6, or a portion thereof.

Preferably, a polypeptide of the invention comprises SNAP-25 or a portion thereof, more preferably full-length SNAP-25.

BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin 1, syntaxin 2, and syntaxin 3. BoNT/X has been found to cleave SNAP-25, VAMP1, VAMP2, VAMP3, VAMP4, VAMP5, Ykt6, and syntaxin 1.

The term “portion thereof” in reference to the substrate of a clostridial neurotoxin includes the site at which the clostridial neurotoxin cleaves, and may further include a number of amino acid residues surrounding said site, e.g. if said further amino acid residues are necessary for cleavage of the substrate by the clostridial neurotoxin cleavage. For example, a fragment may be 25 or 15, amino acids of a clostridial neurotoxin substrate.

A polypeptide of the invention may comprise one or more polypeptides having at least 70% sequence identity to SEQ ID NOs: 4, 6, 8, 10, and/or 12. In one embodiment a polypeptide of the invention comprises one or more polypeptides having at least 80% or 90% sequence identity to SEQ ID NOs: 4, 6, 8, 10, and/or 12. Preferably, a polypeptide of the invention comprises one or more polypeptides shown as SEQ ID NOs: 4, 6, 8, 10, and/or 12.

A polypeptide of the invention may comprise polypeptides having at least 70% sequence identity to SEQ ID NO: 4, SEQ ID NO: 10, and SEQ ID NO: 12. In one embodiment a polypeptide of the invention comprises polypeptides having at least 80% or 90% sequence identity to SEQ ID NO: 4, SEQ ID NO: 10, and SEQ ID NO: 12. Preferably, a polypeptide of the invention comprises polypeptides shown as SEQ ID NO: 4, SEQ ID NO: 10, and SEQ ID NO: 12.

A polypeptide of the invention may comprise a polypeptide sequence having at least 70% sequence identity to SEQ ID NO: 2. In one embodiment a polypeptide of the invention comprises a polypeptide sequence having at least 80% or 90% sequence identity to SEQ ID NO: 2. Preferably a polypeptide of the invention comprises (more preferably consists of) a polypeptide sequence shown as SEQ ID NO: 2.

A nucleotide sequence of the invention (e.g. that encodes a polypeptide sequence of the invention) may comprise one or more nucleotide sequences having at least 70% sequence identity to SEQ ID NOs: 3, 5, 7, 9, and/or 11. In one embodiment nucleotide sequence of the invention comprises one or more nucleotide sequences having at least 80% or 90% sequence identity to SEQ ID NOs: 3, 5, 7, 9, and/or 11. Preferably, a nucleotide sequence of the invention comprises one or more nucleotide sequences shown as SEQ ID NOs: 3, 5, 7, 9, and/or 11.

A nucleotide sequence of the invention may comprise one or more nucleotide sequences having at least 70% sequence identity to SEQ ID NOs: 3, 9, and/or 11. In one embodiment nucleotide sequence of the invention comprises one or more nucleotide sequences having at least 80% or 90% sequence identity to SEQ ID NOs: 3, 9, and/or 11. Preferably, a nucleotide sequence of the invention comprises one or more nucleotide sequences shown as SEQ ID NOs: 3, 9, and/or 11.

A nucleotide sequence of the invention may comprise a nucleotide sequence having at least 70% sequence identity to SEQ ID NO: 1. In one embodiment nucleotide sequence of the invention comprises a nucleotide sequence having at least 80% or 90% sequence identity to SEQ ID NO: 1. Preferably, a nucleotide sequence of the invention comprises (more preferably consists of) a nucleotide sequence shown as SEQ ID NO: 1.

As mentioned above, the invention provides methods for identifying a regulator (e.g. gene or agent) of clostridial neurotoxin activity. The regulation of clostridial neurotoxin activity may be upregulation/positive regulation (e.g. increased clostridial neurotoxin activity) or downregulation/negative regulation of clostridial neurotoxin activity (e.g. decreased clostridial neurotoxin activity). For example, regulation of clostridial neurotoxin activity may be direct regulation at the level of:
i. binding of the clostridial neurotoxin to the cell;
ii. internalisation of the clostridial neurotoxin;
iii. translocation of the clostridial neurotoxin L-chain out of the endosome (including reduction of the disulphide linkage between the L-chain and H-chain);
iv. catalysis (e.g. inhibition or activation of SNARE cleavage); or
v. duration of effect (e.g. persistence of the L-chain activity within the cell cytoplasm).

In other embodiments regulation of clostridial neurotoxin activity may be indirect regulation. Indirect regulation may include regulation of a pathway required for clostridial neurotoxin activity. An example of indirect regulation is regulation of the cellular trafficking of a clostridial neurotoxin receptor, such as Synaptic Vesicle Glycoprotein 2A (SV2).

Preferably regulation of clostridial neurotoxin activity is direct regulation.

Knowledge of indirect regulators can be used to further validate any genes or agents identified by a method of the invention. In one embodiment a clostridial neurotoxin receptor (preferably SV2) can be used to determine whether a gene or agent indirectly regulates trafficking of the clostridial neurotoxin receptor, rather than directly regulating clostridial neurotoxin activity (e.g. by way of clostridial neurotoxin trafficking).

Thus, in one embodiment the methods of the invention comprise further validating a gene or agent identified in a method of the invention, the validation further comprising detecting the presence or absence of a clostridial neurotoxin receptor of the cell when expression of a target gene has been altered in the cell or when the cell has been contacted with an agent. A suitable validation method is provided in Example 5 herein.

The amount of clostridial neurotoxin receptor detected may be compared to a negative control in which the expression of the target gene has not been altered or wherein the cell has not been contacted with the agent. Alternatively or additionally, a negative control may include cells that have not been contacted with a clostridial neurotoxin. When a cell has not been contacted with a clostridial neurotoxin, internalisation and subsequent recycling of the clostridial neurotoxin receptor to the cell surface occurs. In contrast, when a cell has been contacted with a clostridial neurotoxin, cleavage of SNARE proteins by said clostridial neurotoxin prevents efficient recycling of the receptor to the cell surface.

Thus, in one embodiment the amount of clostridial neurotoxin receptor detected is less than the amount detected on the surface of a cell that has not been contacted with the clostridial neurotoxin.

In one embodiment detecting a decreased amount of clostridial neurotoxin receptor on the surface of a cell: a) in which expression of a target gene has been altered; and b) that has been contacted with a clostridial neurotoxin, may indicate that the target gene indirectly regulates clostridial neurotoxin activity.

The decrease referred to above may be when compared to an equivalent cell in which the expression of the target gene is unaltered.

By further employing the use of a known direct inhibitor of clostridial neurotoxin activity, it can be confirmed that the target gene indirectly regulates clostridial neurotoxin activity at the level of clostridial neurotoxin receptor trafficking. For example, indirect regulation may be confirmed if the amount of cell surface receptor detected in the presence of the inhibitor is less than the amount of receptor detected in an equivalent cell (in which the expression of the target gene is unaltered) that has also been contacted with the clostridial neurotoxin and inhibitor (and vice versa).

In one embodiment detecting an equivalent or greater amount of clostridial neurotoxin receptor on the surface of a cell: a) in which expression of a target gene has been altered, and b) that has been contacted with clostridial neurotoxin, may indicate that the target gene does not indirectly regulate clostridial neurotoxin activity.

The equivalent or greater amount referred to above may be when compared to an equivalent cell in which the expression of the target gene is unaltered.

Similarly, in one embodiment detecting a decreased amount of clostridial neurotoxin receptor in a cell contacted with: a) a clostridial neurotoxin, and b) an agent, may indicate that the agent indirectly regulates clostridial neurotoxin activity.

The decreased amount referred to above may be when compared to an equivalent cell in the absence of the agent.

By further employing the use of a known direct inhibitor of clostridial neurotoxin activity, it can be confirmed that the agent indirectly regulates clostridial neurotoxin activity at the level of clostridial neurotoxin receptor trafficking. For example, indirect regulation may be confirmed if the amount of receptor detected in the presence of the inhibitor is less than the amount of receptor detected in an equivalent cell that has also been contacted with the clostridial neurotoxin and inhibitor but not contacted with the agent (and vice versa).

In one embodiment detecting an equivalent or greater amount of clostridial neurotoxin receptor on the surface of a cell contacted with: a) a clostridial neurotoxin, and b) an agent, may indicate that the agent does not indirectly regulate clostridial neurotoxin activity.

The equivalent or greater amount referred to above may be when compared to an equivalent cell in the absence of the agent.

Preferably the clostridial neurotoxin receptor is SV2.

The present invention overcomes a limitation of previous assays that, in many cell types, the normal levels of cell surface SV2 are low and difficult to detect and it is therefore unfeasible/difficult to determine if those levels are changed when expression of a target gene is altered or in the presence of an agent.

In one embodiment a known direct inhibitor of clostridial neurotoxin activity is an siRNA or shRNA that downregulates thioredoxin reductase expression.

The methods of the present invention are particularly suited for high throughput screening. In this regard, the methods may comprise the use of a plurality of samples of cells, preferably wherein the expression of a different target gene is altered in each sample of cells. RNAi libraries (or equivalent gene silencing libraries) are particularly well-suited for use in such high throughput methods, as are drug libraries.

Thus, in one embodiment a method of the invention comprises:
a. providing a plurality of samples of human neuronal cells expressing a polypeptide that comprises a C-terminal detectable label, wherein the polypeptide is cleavable by a clostridial neurotoxin;
b. altering expression of a different target gene in each of the plurality of samples, wherein the target gene is different in each sample of cells;
c. contacting the cells with a clostridial neurotoxin;
d. measuring the amount of C-terminal detectable label, thereby quantifying clostridial neurotoxin activity; and
e. identifying one or more target genes as regulators of clostridial neurotoxin activity when the quantified clostridial neurotoxin activity is different to the quantified clostridial neurotoxin activity when expression of the one or more target genes is unaltered; or
f. identifying that one or more target genes are not regulators of clostridial neurotoxin activity when the quantified clostridial neurotoxin activity is equivalent to the quantified clostridial neurotoxin activity when expression of the one or more target genes is unaltered.

In another embodiment the method comprises:
a. providing a plurality of samples of human neuronal cells expressing a polypeptide that comprises a C-terminal detectable label, wherein the polypeptide is cleavable by a clostridial neurotoxin;
b. contacting the cells with a clostridial neurotoxin and an agent, wherein the contacting is sequential or simultaneous, and wherein each of the samples is contacted with a different agent;
c. measuring the amount of C-terminal detectable label, thereby quantifying clostridial neurotoxin activity; and
d. identifying one or more agents as regulators of clostridial neurotoxin activity when the quantified clostridial neurotoxin activity is different to the quantified clostridial neurotoxin activity in the absence of the one or more agents; or
e. identifying that one or more agents are not regulators of clostridial neurotoxin activity when the quantified clostridial neurotoxin activity is equivalent to the quantified clostridial neurotoxin activity in the absence of the one or more agents.

The term “plurality” as used herein means two or more. Preferably the term “plurality” means more than two, such as 200, 250 or 300.

The method may comprise the use of a multi-well plate, wherein each well contains one of the plurality of samples. The sensitivity and high signal-to-noise ratio of the methods of the present invention allow for the use of multi-well plates comprising ≥150 wells (preferably 300 wells, such as a 384 well plate). Advantageously, this allows for improved throughput when compared to methods that employ <150 well plates (such as 96 well plates).

Quantifying clostridial neurotoxin activity by measuring the amount of C-terminal detectable label according to the methods of the invention is particularly well suited for easy and rapid imaging and quantification using automated and/or high-throughput means (e.g. microscopy coupled with automated analysis software). Moreover, the human neuronal cells of the invention are highly sensitive to clostridial neurotoxins, thus allowing for shorter exposure times while generating sufficient signal for detection. This is especially suited for use in automated screening with smaller multi-well plate formats, e.g. comprising ≥150 wells (such as 384 well plates) since shorter incubation times make the logistics of scheduling automated steps in the method less complicated. The severity of bottlenecks in the progression of the train of multiple plates (in a screening run) from one stage to the next, where some stages are completed in minutes and others over hours or days, is reduced; fewer and shorter hold steps are required, and the equipment is occupied for less time.

The methods for identifying a gene that regulates clostridial neurotoxin activity comprise altering expression of a target gene. The alteration may be an upregulation or a downregulation of expression (compared to the expression level in an equivalent cell in which expression has not been altered, i.e. where expression of the target gene is “unaltered”). Altering expression of a target gene may be achieved using any method known in the art, for example by way of gene editing, overexpression or gene silencing.

In one embodiment expression is altered by downregulating expression of the target gene (e.g. by way of gene silencing). A preferred method for downregulating expression of a target gene is by RNA interference (RNAi), e.g. using short interfering or short hairpin RNAs (siRNAs or shRNAs).

In embodiments where expression is altered by downregulating expression of the target gene, a target gene may be identified as a positive regulator of clostridial neurotoxin activity when the quantified clostridial neurotoxin activity is less than the quantified clostridial neurotoxin activity when expression of the target gene is unaltered. Alternatively, a target gene may be identified as a negative regulator of clostridial neurotoxin activity when the quantified clostridial neurotoxin activity is greater than the quantified clostridial neurotoxin activity when expression of the target gene is unaltered.

In contrast, in embodiments where expression is altered by upregulating expression of the target gene, a target gene may be identified as a negative regulator of clostridial neurotoxin activity when the quantified clostridial neurotoxin activity is less than the quantified clostridial neurotoxin activity when expression of the target gene is unaltered. Alternatively, a target gene may be identified as a positive regulator of clostridial neurotoxin activity when the quantified clostridial neurotoxin activity is greater than the quantified clostridial neurotoxin activity when expression of the target gene is unaltered.

The methods for identifying an agent that regulates clostridial neurotoxin activity comprise contacting the cells with a clostridial neurotoxin and an agent, wherein the contacting is sequential (e.g. contacting with a clostridial neurotoxin and then an agent, or contacting with an agent and then a clostridial neurotoxin) or simultaneous. Preferably the contacting is sequential.

In one embodiment the cells are contacted with an agent before being contacted with a clostridial neurotoxin. Such methods are particularly suitable for identifying agents capable of preventing intoxication by a clostridial neurotoxin. Agents capable of preventing intoxication may be suitable for use in therapy as prophylactics.

Alternatively, the cells may be contacted with an agent after being contacted with a clostridial neurotoxin. Such methods are particularly suitable for identifying agents capable of inhibiting clostridial neurotoxin activity post-intoxication, and may be suitable for use in therapy as post-intoxication therapeutics.

An agent identified as a positive regulator of clostridial neurotoxin activity may be a clostridial neurotoxin sensitising agent. Such agents may be used in therapy in combination with a clostridial neurotoxin to modulate local activity of clostridial neurotoxins (e.g. to allow reduced dosage and minimise spread to other tissues).

Clostridial neurotoxin activity can be quantified by measuring the amount of C-terminal detectable label. Preferably clostridial neurotoxin activity can be quantified by measuring the amount of a C-terminal detectable label and an N-terminal detectable label.

The amount of C-terminal detectable label after contacting the cells with clostridial neurotoxin may be compared to the amount of C-terminal detectable label measured in the absence of clostridial neurotoxin. For example, the amount of C-terminal detectable label may be measured in a sample of the same cells before and after contacting with clostridial neurotoxin. Alternatively, the amount of C-terminal detectable label may be measured after contacting with clostridial neurotoxin and compared to the amount of C-terminal detectable label present in an equivalent sample of cells under equivalent conditions, which have not been contacted with the clostridial neurotoxin.

In one embodiment loss of the C-terminal detectable label (e.g. over time) when compared to an equivalent cell that has not been contacted with clostridial neurotoxin or when compared to the same cell before contacting with clostridial neurotoxin indicates the presence of clostridial neurotoxin activity. Alternatively, in one embodiment no loss (or detection of an equivalent amount) of C-terminal detectable label (e.g. over time) when compared to an equivalent cell that has not been contacted with clostridial neurotoxin or when compared to the same cell before contacting with clostridial neurotoxin indicates the absence of clostridial neurotoxin activity. In one embodiment a partial loss of C-terminal detectable label (e.g. over time) when compared to an equivalent cell that has not been contacted with clostridial neurotoxin or when compared to the same cell before contacting with clostridial neurotoxin may indicate the presence of reduced clostridial neurotoxin activity (and vice versa).

The method may further comprise measuring the amount of N-terminal detectable label. Similarly to the C-terminal label, the amount of N-terminal detectable label after contacting the cells with clostridial neurotoxin may be compared to the amount of N-terminal detectable label measured in the absence of clostridial neurotoxin. For example, the amount of N-terminal detectable label may be measured in a sample of the same cells before and after contacting with clostridial neurotoxin. Alternatively, the amount of N-terminal detectable label may be measured after contacting with clostridial neurotoxin and compared to the amount of N-terminal detectable label present in an equivalent sample of cells under equivalent conditions, which have not been contacted with the clostridial neurotoxin.

Preferably clostridial neurotoxin activity is determined by comparing the amount of N-terminal label and C-terminal label. A greater amount of N-terminal label to C-terminal label preferably indicates that the polypeptide has been cleaved by the clostridial neurotoxin.

A detectable label of the invention may be measured at one or more time points after contacting the cells with clostridial neurotoxin. By doing so, clostridial neurotoxin activity rates may be calculated.

In one embodiment the detectable label may be measured after contacting the cells with clostridial neurotoxin for at least 2, 5, 10, 15, 20, 30, 40, 50, 60 or 70 hours. In other embodiments the detectable label may be measured after contacting the cells with clostridial neurotoxin for less than 100, 80, 70, 60 or 50 hours. Preferably, the detectable label may be measured after contacting the cells with clostridial neurotoxin for less than 72 hours, more preferably less than 50 hours. Thus, the detectable label may be measured after contacting the cells with clostridial neurotoxin for 20-60 hours, preferably 40-55 hours (e.g. about 48 hours). The cells may be fixed prior to measuring the amount of the detectable label.

When comparing the cells of the method of the invention with a control (e.g. an equivalent cell in which expression of a target gene is unaltered or that has not been contacted with an agent of the invention or another control referred to above) it is envisaged that the measurements are performed in the same way (e.g. at the same time point after contacting the cells with clostridial neurotoxin). This allows comparisons between the quantified clostridial neurotoxin activity of the cells of the method and a control in order to identify the presence or absence of a difference in clostridial neurotoxin activity.

A measuring or detecting step of the invention may be carried out using any suitable means known to the skilled person. In one embodiment a fluorescent label present on a polypeptide (or present on an antibody binding to a clostridial neurotoxin receptor) is excited with a suitable wavelength of light, and resultant fluorescence detected. Thus, the invention may employ the use of fluorescence microscopy. In a preferred embodiment the measuring or detection step comprises the use of a high-throughput screening system. An example of a suitable system is the Opera Phenix™ High-Content Screening System, which is commercially available from PerkinElmer and/or the use of suitable imaging software, such as Columbus™ software (commercially available form PerkinElmer). In some embodiments the measuring or detection step of the invention is automated, and may employ the use of robotics.

A method of the invention preferably does not employ the use of electronic coupling between the detectable labels described herein. In particular it is preferred that the labels are not positioned such that a donor label (e.g. an N-terminal label) can transfer energy to an acceptor label (e.g. a C-terminal label), such as by a dipole-dipole coupling mechanism. Preferably the invention does not employ the use of Förster Resonance Energy Transfer (FRET).

The methods of the invention may comprise a validation step in which the quantified clostridial neurotoxin activity detected in the method is compared to a positive control. Positive validation may occur when the quantified clostridial neurotoxin activity detected is equivalent to that of the positive control.

A method of the invention may comprise further validating that a regulator is indeed a positive regulator. In one embodiment the method comprises contacting the cells with a known negative regulator of clostridial neurotoxin activity and showing that the effect on clostridial neurotoxin activity can be reversed. In one embodiment the method comprises upregulating expression of a known negative regulator of clostridial neurotoxin activity and showing that the effect on clostridial neurotoxin activity can be reversed. Preferably, the method comprises downregulating expression of a known positive regulator of clostridial neurotoxin activity and showing that the effect on clostridial neurotoxin activity can be reversed.

A method of the invention may comprise further validating that a regulator is indeed a negative regulator. In one embodiment the method comprises contacting the cells with a known positive regulator of clostridial neurotoxin activity and showing that the effect on clostridial neurotoxin activity can be reversed. In one embodiment the method comprises downregulating expression of a known negative regulator of clostridial neurotoxin activity and showing that the effect on clostridial neurotoxin activity can be reversed. In one embodiment the method comprises upregulating expression of a known positive regulator of clostridial neurotoxin activity and showing that the effect on clostridial neurotoxin activity can be reversed.

A known (positive) regulator of clostridial neurotoxin activity is thioredoxin reductase. In one embodiment expression of thioredoxin reductase is upregulated. Preferably expression of thioredoxin reductase is downregulated.

The term “equivalent” as used herein may mean that the two or more values being compared are not statistically significantly different. Preferably the term “equivalent” as used herein means that the two or more values are identical. Similarly the term “unaltered” as used herein may mean that the two or more values being compared are not statistically significantly different. Preferably the term “unaltered” as used herein means that the two or more values are identical.

A difference or alteration referred to herein (e.g. an increase or decrease) preferably means a statistically significantly difference or alteration (e.g. a statistically significant increase or decrease). Thus, a difference in quantified clostridial neurotoxin activity is preferably a statistically significant difference in quantified clostridial neurotoxin activity. In one embodiment a difference in quantified clostridial neurotoxin activity is a difference of at least 1%, 5%, 10%, 15%, 20%, 25%, 30%, 40% or 50% when compared to the quantified clostridial neurotoxin activity of a negative control (e.g. an equivalent cell in which expression of a target gene is unaltered or that has not been contacted with an agent of the invention or another control referred to above).

The methods of the present invention are in vitro methods.

In one embodiment the methods of the invention are live cell methods and optionally the methods employ real-time monitoring of clostridial neurotoxin activity when expression of a target gene is altered and/or when the cells are contacted with an agent.

Preferably, the term “contacting a cell with a clostridial neurotoxin” means that a surface of the cell is contacted with a clostridial neurotoxin. Suitably, a clostridial neurotoxin may be added to a medium in which the cell is present, for example a cell culture medium. The term “contacting a cell with a clostridial neurotoxin” preferably excludes contacting by expressing a clostridial neurotoxin in the cell, which technique provides no information regarding binding, internalisation, and/or translocation of the clostridial neurotoxin.

A method of the invention typically comprises contacting the cells with less than 1500 nM clostridial neurotoxin. In one embodiment the cells are contacted with less than 1000 nM, such as less than 500 nM, 250 nM or 100 nM clostridial neurotoxin.

A method of the invention may comprise contacting the cells with at least 1 nM, 5 nM, 10 nM, 20 nM or 50 nM clostridial neurotoxin.

In one embodiment a method of the invention may comprise contacting the cells with 1-1000 nM clostridial neurotoxin, such as 1-500 nM or 1-200 nM clostridial neurotoxin.

The inventors have surprisingly found that by contacting the cells with a buffer comprising GDNF and cell-permeable cAMP (and optionally further comprising CaCl₂, and KCl), that the sensitivity of the methods of the invention can be improved. In the presence of the buffer, the cells are highly sensitive to clostridial neurotoxin at a concentration of less than 100 nM, preferably less than 50 nM, more preferably at 5-15 nM (e.g. ˜10 nM).

The buffer may comprise GDNF, d-cAMP, CaCl₂, and KCl. GDNF may be present at 1-100 ng/ml, preferably 10 ng/ml. d-cAMP may be present at 0.1-5 mM, preferably 1 mM. CaCl₂ may be present at 0.1-7 mM, preferably 2 mM. KCl may be present at 1-100 mM, preferably 56 mM.

The buffer may be a component of a kit of the invention.

Thus, in one aspect the invention provides a composition comprising:
a. a clostridial neurotoxin; and
b. a buffer, the buffer comprising GDNF, cell-permeable cyclic adenosine monophosphate (cAMP), CaCl₂ and KCl.

In one embodiment the composition comprises GDNF present at 1-100 ng/ml, d-cAMP present at 0.1-5 mM, CaCl₂ present at 0.1-7 mM, and KCl present at 1-100 mM. Preferably the buffer comprises GDNF present at 10 ng/ml, d-cAMP present at 1 mM, CaCl₂ present at 2 mM, and KCl present at 56 mM.

Reference herein to “cAMP” is preferably interchangeable with d-cAMP.

The composition may comprise less than 1500 nM clostridial neurotoxin. In one embodiment the composition comprises less than 1000 nM, such as less than 500 nM, 250 nM or 100 nM clostridial neurotoxin. In some embodiments the composition may comprise at least 1 nM, 5 nM, 10 nM, 20 nM or 50 nM clostridial neurotoxin. In one embodiment the composition comprises 1-1000 nM clostridial neurotoxin, such as 1-500 nM or 1-200 nM clostridial neurotoxin. Preferably, the composition comprises a clostridial neurotoxin at a concentration of less than 100 nM, preferably less than 50 nM, more preferably at 5-15 nM (e.g. ˜10 nM).

In some embodiments the composition further comprises cells, such as human neuronal cells described herein.

The cells may be incubated with clostridial neurotoxin for any suitable time. In one embodiment the cells may be incubated with clostridial neurotoxin for at least 2, 5, 10, 15, 20, 30, 40, 50, 60 or 70 hours. In other embodiments the cells may be incubated with clostridial neurotoxin for less than 100, 80, 70, 60 or 50 hours. Preferably, the cells are incubated with clostridial neurotoxin for less than 72 hours, more preferably less than 50 hours. Thus, the cells may be incubated with clostridial neurotoxin for 20-60 hours, preferably 40-55 hours (e.g. about 48 hours). Advantageously, the human neuronal cells of the present invention are highly sensitive to clostridial neurotoxins thereby allowing for short incubation periods with clostridial neurotoxin of less than 72 hours (˜48 hours), and thus reducing the time needed to carry out the assay.

The cells may be incubated with clostridial neurotoxin at any suitable temperature. In one embodiment the cells are incubated with clostridial neurotoxin at 30-40° C., preferably 37° C.

A target gene or agent identified by a method of the invention may be used in a method for treating a disorder. Thus, in one aspect the invention provides a method for treating a disorder comprising administering to a subject an agent identified by a method of the invention. In another aspect the invention provides a method for treating a disorder comprising altering expression of a gene in a subject, wherein the gene has been identified by a method of the invention.

In one aspect the invention provides a kit comprising: a cell according to the invention; and optionally instructions for use of the same (e.g. in a method described herein). In a related aspect the invention provides a kit comprising a nucleotide sequence according to the invention; and optionally instructions for use of the same (e.g. in a method described herein). In a related aspect the invention provides a kit comprising a vector according to the invention; and optionally instructions for use of the same (e.g. in a method described herein). In one embodiment a kit comprises a cell (preferably a cell identical to the cell type of the present invention but not comprising a nucleotide sequence of the invention and not expressing a polypeptide of the invention), a nucleotide sequence or vector of the invention, and optionally instructions for use of the same (e.g. in a method described herein). Said kits may comprise one or more separate containers, each containing a recited constituent of the kit.

The present invention is suitable for application to many different varieties of clostridial neurotoxin. Thus, in the context of the present invention, the term “clostridial neurotoxin” embraces toxins produced by C. botulinum (botulinum neurotoxin serotypes A, B, C1, D, E, F, G, H, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing. The term “clostridial neurotoxin” also embraces botulinum neurotoxin serotype H.

Botulinum neurotoxin (BoNT) is produced by C. botulinum in the form of a large protein complex, consisting of BoNT itself complexed to a number of accessory proteins. There are at present nine different classes of botulinum neurotoxin, namely: botulinum neurotoxin serotypes A, B, C1, D, E, F, G, H, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine.

Tetanus toxin is produced in a single serotype by C. tetani. C. butyricum produces BoNT/E, while C. baratii produces BoNT/F.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, in one embodiment, the clostridial neurotoxin of the invention is a modified clostridial neurotoxin, or a modified clostridial neurotoxin derivative, or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL (SEQ ID NO: 22), xExxxLL (SEQ ID NO: 23), xExxxIL (SEQ ID NO: 24), and xExxxLM (SEQ ID NO: 25) (wherein x is any amino acid). Suitable tyrosine-based motifs include Y-x-x-Hy (SEQ ID NO: 26) (wherein Hy is a hydrophobic amino acid). Examples of modified clostridial neurotoxins comprising leucine- and tyrosine-based motifs are described in WO 2002/08268, which is hereby incorporated by reference in its entirety.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to patients who are immunologically resistant to a given clostridial neurotoxin subtype, to patients who may have a lower than average concentration of receptors to a given clostridial neurotoxin heavy chain binding domain, or to patients who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the clostridial neurotoxin of the invention is an hybrid clostridial neurotoxin, or an chimeric clostridial neurotoxin.

The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OT022244.1), which cleaves VAMP2 and SNAP25, and the Chryseobacterium pipero encoded toxin (NCBI Ref.Seq: WP_034687872.1).

In a preferred embodiment a clostridial neurotoxin is a botulinum neurotoxin, more preferably BoNT/A.

In one embodiment the clostridial neurotoxin may be BoNT/A. A reference BoNT/A sequence is shown as SEQ ID NO: 13.

In another embodiment the clostridial neurotoxin may be BoNT/B. A reference BoNT/B sequence is shown as SEQ ID NO: 14.

In another embodiment the clostridial neurotoxin may be BoNT/C. A reference BoNT/C₁ sequence is shown as SEQ ID NO: 15.

In another embodiment the clostridial neurotoxin may be BoNT/D. A reference BoNT/D sequence is shown as SEQ ID NO: 16.

In another embodiment the clostridial neurotoxin may be BoNT/E. A reference BoNT/E sequence is shown as SEQ ID NO: 17.

In another embodiment the clostridial neurotoxin may be BoNT/F. A reference BoNT/F sequence is shown as SEQ ID NO: 18.

In another embodiment the clostridial neurotoxin may be BoNT/G. A reference BoNT/G sequence is shown as SEQ ID NO: 19.

In one embodiment the clostridial neurotoxin may be BoNT/X. A reference BoNT/X sequence is shown as SEQ ID NO: 20.

In another embodiment the clostridial neurotoxin may be TeNT. A reference TeNT sequence is shown as SEQ ID NO: 21.

Embodiments related to the various methods of the invention are intended to be applied equally to other methods, the cells, polypeptides, nucleotide sequences, kits, and compositions of the invention, and vice versa.

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative Amino Acid Substitutions
Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows a method for generating ReNcell VM stably expressing TagRFPT-SNAP25-TagGFP. The images (lower) show red fluorescence (right) and green fluorescence (right).

FIG. 2 shows BoNT/A-induced degradation of the C-terminal fragment of the construct. ReD SNAPR cells were differentiated for 14 days without growth factors and 100 nM of BoNT/A was then added to the cells and intoxicated for 48 hours. (A) Cells were imaged using fluorescence microscopy and the corresponding GFP, RFP and GFP/RFP merged channels are as shown. (B) Lysates were subjected to SDS-PAGE for western blot analysis. Rabbit antibodies against tagRFPT and tagGFP were used for the western blot.

FIG. 3 shows a diagram summarising degradation of the construct by BoNT/A.

FIG. 4 shows that the enhanced differentiation and stimulation buffer sensitised ReD SNAPR cells to BoNT/A. (A) ReD SNAPR cells were subjected to ReNcell differentiation media supplemented with and without GDNF and d-cAMP during differentiation and high potassium buffer during intoxication. The resulting modified media is known as ReDS (ReNcell enhanced differentiation and stimulation) media. The loss of fluorescence of tagGFP upon cleavage of the dual-tagged SNAP25 construct was observed in a dose-dependent manner. ReD SNAPR cells in ReDS media exhibited enhanced sensitivity towards BoNT/A as compared to normal ReNcell media as detected using confocal microscopy. (B) Quantification of BoNT/A-mediated cleavage in both conditions showed the EC₅₀ was improved from 43 nM to 6 nM in ReD SNAPR exposed to ReDS media. (C) Western blot detection of BoNT/A-mediated SNAP25 construct cleavage in ReD SNAPR cells in normal and ReDS media. The upper blot shows cell lysates probed with tRFP antibody and the lower blot probed with tGFP antibody.

FIG. 5 shows siRNA against TrxR rescues BoNT/A-mediated construct cleavage. Graphs of TrxR1 knockdown levels and construct cleavage are shown. The upper panel (Red SNAPR) shows green and red fluorescence (siNT3, −BoNT/A), red fluorescence only (siNR3, +BoNT/A), green and red fluorescence (siTrxR, −BoNT/A), and green and red fluorescence (siTrxR, −BoNT/A). The lower panel shows staining for TrxR1 in the presence of siNT3 (for both −BoNT/A and +BoNT/A conditions) and the absence of staining for TrxR1 in the presence of siTrxR (for both −BoNT/A and +BoNT/A conditions).

FIG. 6 shows that BoNT/A intoxication prevents trafficking of SV2 to the cell surface A) ReNcell VM cells were treated with siNT3, siVAMP2 and siTrxR1 for 72 hours before addition of BoNT/A. Cells were fixed and stained with a primary antibody against SV2A (without permeabilisation). An Alexa-488 secondary antibody was used against the primary antibody. (B) An illustration of BoNT/A-mediated decreased trafficking of SV2 to the cell surface.

FIG. 7 shows a schematic for performing a genome-wide siRNA screen using the cell line of the invention.

SEQUENCE LISTING
Where an initial Met amino acid residue or a corresponding initial codon is
indicated in any of the following SEQ ID NOs, said residue/codon may be
optional.
(Nucleotide Sequence of Construct TaqRFPT-SNAP25-TaqGFP)
SEQ ID NO: 1
ATGGTGTCTAAGGGCGAAGAGCTGATTAAGGAGAACATGCACATGAAGCTGTACATGGAGGGCACCGTGAACAAC
CACCACTTCAAGTGCACATCCGAGGGCGAAGGCAAGCCCTACGAGGGCACCCAGACCATGAGAATCAAGGTGGTC
GAGGGCGGCCCTCTCCCCTTCGCCTTCGACATCCTGGCTACCAGCTTCATGTACGGCAGCAGAACCTTCATCAAC
CACACCCAGGGCATCCCCGACTTCTTTAAGCAGTCCTTCCCTGAGGGCTTCACATGGGAGAGAGTCACCACATAC
GAAGACGGGGGCGTGCTGACCGCTACCCAGGACACCAGCCTCCAGGACGGCTGCCTCATCTACAACGTCAAGATC
AGAGGGGTGAACTTCCCATCCAACGGCCCTGTGATGCAGAAGAAAACACTCGGCTGGGAGGCCAACACCGAGATG
CTGTACCCCGCTGACGGCGGCCTGGAAGGCAGAACCGACATGGCCCTGAAGCTCGTGGGCGGGGGCCACCTGATC
TGCAACTTCAAGACCACATACAGATCCAAGAAACCCGCTAAGAACCTCAAGATGCCCGGCGTCTACTATGTGGAC
CACAGACTGGAAAGAATCAAGGAGGCCGACAAAGAGACCTACGTCGAGCAGCACGAGGTGGCTGTGGCCAGATAC
TGCGACCTCCCTAGCAAACTGGGGCACAAACTTAATGGCATGGACGAGCTGTACAAGGGCTCGGGCTCGGGCTCG
GGCGTGGCCGAAGACGCAGACATGCGCAATGAGCTGGAGGAGATGCAGCGAAGGGCTGACCAGTTGGCTGATGAG
TCGCTGGAAAGCACCCGTCGTATGCTGCAACTGGTTGAAGAGAGTAAAGATGCTGGTATCAGGACTTTGGTTATG
TTGGATGAACAAGGAGAACAACTCGATCGTGTCGAAGAAGGCATGAACCATATCAACCAAGACATGAAGGAGGCT
GAGAAAAATTTAAAAGATTTAGGGAAATGCTGTGGCCTTTTCATATGTCCTTGTAACAAGCTTAAATCAAGTGAT
GCTTACAAAAAAGCCTGGGGCAATAATCAGGACGGAGTGGTGGCCAGCCAGCCTGCTCGTGTAGTGGACGAACGG
GAGCAGATGGCCATCAGTGGCGGCTTCATCCGCAGGGTAACAAATGATGCCCGAGAAAATGAAATGGATGAAAAC
CTAGAGCAGGTGAGCGGCATCATCGGGAACCTCCGTCACATGGCCCTGGATATGGGCAATGAGATCGATACACAG
AATCGCCAGATCGACAGGATCATGGAGAAGGCTGATTCCAACAAAACCAGAATTGATGAGGCCAACCAACGTGCA
ACAAAGATGCTGGGAAGTGGTTACGGCGGCTCGGGCTCGGGCGTGAGCGGGGGCGAGGAGCTGTTCGCCGGCATC
GTGCCCGTGCTGATCGAGCTGGACGGCGACGTGCACGGCCACAAGTTCAGCGTGCGCGGCGAGGGCGAGGGCGAC
GCCGACTACGGCAAGCTGGAGATCAAGTTCATCTGCACCACCGGCAAGCTGCCCGTGCCCTGGCCCACCCTGGTG
ACCACCCTCTGCTACGGCATCCAGTGCTTCGCCCGCTACCCCGAGCACATGAAGATGAACGACTTCTTCAAGAGC
GCCATGCCCGAGGGCTACATCCAGGAGCGCACCATCCAGTTCCAGGACGACGGCAAGTACAAGACCCGCGGCGAG
GTGAAGTTCGAGGGCGACACCCTGGTGAACCGCATCGAGCTGAAGGGCAAGGACTTCAAGGAGGACGGCAACATC
CTGGGCCACAAGCTGGAGTACAGCTTCAACAGCCACAACGTGTACATCCGCCCCGACAAGGCCAACAACGGCCTG
GAGGCTAACTTCAAGACCCGCCACAACATCGAGGGCGGCGGCGTGCAGCTGGCCGACCACTACCAGACCAACGTG
CCCCTGGGCGACGGCCCCGTGCTGATCCCCATCAACCACTACCTGAGCACTCAGACCAAGATCAGCAAGGACCGC
AACGAGGCCCGCGACCACATGGTGCTCCTGGAGTCCTTCAGCGCCTGCTGCCACACCCACGGCATGGACGAGCTG
TACAGGTAA
(Polypeptide Sequence of Construct TaqRFPT-SNAP25-TaqGFP)
SEQ ID NO: 2
MVSKGEELIKENMHMKLYMEGTVNNHHFKCTSEGEGKPYEGTQTMRIKVVEGGPLPFAFDILATSFMYGSRTFIN
HTQGIPDFFKQSFPEGFTWERVTTYEDGGVLTATQDTSLQDGCLIYNVKIRGVNFPSNGPVMQKKTLGWEANTEM
LYPADGGLEGRTDMALKLVGGGHLICNFKTTYRSKKPAKNLKMPGVYYVDHRLERIKEADKETYVEQHEVAVARY
CDLPSKLGHKLNGMDELYKGSGSGSGVAEDADMRNELEEMQRRADQLADESLESTRRMLQLVEESKDAGIRTLVM
LDEQGEQLDRVEEGMNHINQDMKEAEKNLKDLGKCCGLFICPCNKLKSSDAYKKAWGNNQDGVVASQPARVVDER
EQMAISGGFIRRVTNDARENEMDENLEQVSGIIGNLRHMALDMGNEIDTQNRQIDRIMEKADSNKTRIDEANQRA
TKMLGSGYGGSGSGVSGGEELFAGIVPVLIELDGDVHGHKFSVRGEGEGDADYGKLEIKFICTTGKLPVPWPTLV
TTLCYGIQCFARYPEHMKMNDFFKSAMPEGYIQERTIQFQDDGKYKTRGEVKFEGDTLVNRIELKGKDFKEDGNI
LGHKLEYSFNSHNVYIRPDKANNGLEANFKTRHNIEGGGVQLADHYQTNVPLGDGPVLIPINHYLSTQTKISKDR
NEARDHMVLLESFSACCHTHGMDELYR*
(Nucleotide Sequence of TagRFPT)
SEQ ID NO: 3
ATGGTGTCTAAGGGCGAAGAGCTGATTAAGGAGAACATGCACATGAAGCTGTACATGGAGGGCACCGTGAACAAC
CACCACTTCAAGTGCACATCCGAGGGCGAAGGCAAGCCCTACGAGGGCACCCAGACCATGAGAATCAAGGTGGTC
GAGGGCGGCCCTCTCCCCTTCGCCTTCGACATCCTGGCTACCAGCTTCATGTACGGCAGCAGAACCTTCATCAAC
CACACCCAGGGCATCCCCGACTTCTTTAAGCAGTCCTTCCCTGAGGGCTTCACATGGGAGAGAGTCACCACATAC
GAAGACGGGGGCGTGCTGACCGCTACCCAGGACACCAGCCTCCAGGACGGCTGCCTCATCTACAACGTCAAGATC
AGAGGGGTGAACTTCCCATCCAACGGCCCTGTGATGCAGAAGAAAACACTCGGCTGGGAGGCCAACACCGAGATG
CTGTACCCCGCTGACGGCGGCCTGGAAGGCAGAACCGACATGGCCCTGAAGCTCGTGGGCGGGGGCCACCTGATC
TGCAACTTCAAGACCACATACAGATCCAAGAAACCCGCTAAGAACCTCAAGATGCCCGGCGTCTACTATGTGGAC
CACAGACTGGAAAGAATCAAGGAGGCCGACAAAGAGACCTACGTCGAGCAGCACGAGGTGGCTGTGGCCAGATAC
TGCGACCTCCCTAGCAAACTGGGGCACAAACTTAATGGCATGGACGAGCTGTACAAG
(Polypeptide Sequence of TagRFPT)
SEQ ID NO: 4
MVSKGEELIKENMHMKLYMEGTVNNHHFKCTSEGEGKPYEGTQTMRIKVVEGGPLPFAFDILATSFMYGSRTFIN
HTQGIPDFFKQSFPEGFTWERVTTYEDGGVLTATQDTSLQDGCLIYNVKIRGVNFPSNGPVMQKKTLGWEANTEM
LYPADGGLEGRTDMALKLVGGGHLICNFKTTYRSKKPAKNLKMPGVYYVDHRLERIKEADKETYVEQHEVAVARY
CDLPSKLGHKLNGMDELYK
(Nucleotide Sequence of Glycine-Serine rich linker 1)
SEQ ID NO: 5
GGCTCGGGCTCGGGCTCGGGC
(Polypeptide Sequence of Glycine-Serine rich linker 1)
SEQ ID NO: 6
GSGSGSG
(Nucleotide Sequence of Glycine-Serine rich linker 2)
SEQ ID NO: 7
GGCGGCTCGGGCTCGGGC
(Polypeptide Sequence of Glycine-Serine rich linker 2)
SEQ ID NO: 8
GGSGSG
(Nucleotide Sequence of SNAP25)
SEQ ID NO: 9
GTGGCCGAAGACGCAGACATGCGCAATGAGCTGGAGGAGATGCAGCGAAGGGCTGACCAGTTGGCTGATGAGTCG
CTGGAAAGCACCCGTCGTATGCTGCAACTGGTTGAAGAGAGTAAAGATGCTGGTATCAGGACTTTGGTTATGTTG
GATGAACAAGGAGAACAACTCGATCGTGTCGAAGAAGGCATGAACCATATCAACCAAGACATGAAGGAGGCTGAG
AAAAATTTAAAAGATTTAGGGAAATGCTGTGGCCTTTTCATATGTCCTTGTAACAAGCTTAAATCAAGTGATGCT
TACAAAAAAGCCTGGGGCAATAATCAGGACGGAGTGGTGGCCAGCCAGCCTGCTCGTGTAGTGGACGAACGGGAG
CAGATGGCCATCAGTGGCGGCTTCATCCGCAGGGTAACAAATGATGCCCGAGAAAATGAAATGGATGAAAACCTA
GAGCAGGTGAGCGGCATCATCGGGAACCTCCGTCACATGGCCCTGGATATGGGCAATGAGATCGATACACAGAAT
CGCCAGATCGACAGGATCATGGAGAAGGCTGATTCCAACAAAACCAGAATTGATGAGGCCAACCAACGTGCAACA
AAGATGCTGGGAAGTGGTTAC
(Polypeptide Sequence of SNAP25)
SEQ ID NO: 10
VAEDADMRNELEEMQRRADQLADESLESTRRMLQLVEESKDAGIRTLVMLDEQGEQLDRVEEGMNHINQDMKEAE
KNLKDLGKCCGLFICPCNKLKSSDAYKKAWGNNQDGVVASQPARVVDEREQMAISGGFIRRVTNDARENEMDENL
EQVSGIIGNLRHMALDMGNEIDTQNRQIDRIMEKADSNKTRIDEANQRATKMLGSGY
(Nucleotide Sequence of TaqGFP)
SEQ ID NO: 11
GTGAGCGGGGGCGAGGAGCTGTTCGCCGGCATCGTGCCCGTGCTGATCGAGCTGGACGGCGACGTGCACGGCCAC
AAGTTCAGCGTGCGCGGCGAGGGCGAGGGCGACGCCGACTACGGCAAGCTGGAGATCAAGTTCATCTGCACCACC
GGCAAGCTGCCCGTGCCCTGGCCCACCCTGGTGACCACCCTCTGCTACGGCATCCAGTGCTTCGCCCGCTACCCC
GAGCACATGAAGATGAACGACTTCTTCAAGAGCGCCATGCCCGAGGGCTACATCCAGGAGCGCACCATCCAGTTC
CAGGACGACGGCAAGTACAAGACCCGCGGCGAGGTGAAGTTCGAGGGCGACACCCTGGTGAACCGCATCGAGCTG
AAGGGCAAGGACTTCAAGGAGGACGGCAACATCCTGGGCCACAAGCTGGAGTACAGCTTCAACAGCCACAACGTG
TACATCCGCCCCGACAAGGCCAACAACGGCCTGGAGGCTAACTTCAAGACCCGCCACAACATCGAGGGCGGCGGC
GTGCAGCTGGCCGACCACTACCAGACCAACGTGCCCCTGGGCGACGGCCCCGTGCTGATCCCCATCAACCACTAC
CTGAGCACTCAGACCAAGATCAGCAAGGACCGCAACGAGGCCCGCGACCACATGGTGCTCCTGGAGTCCTTCAGC
GCCTGCTGCCACACCCACGGCATGGACGAGCTGTACAGGTAA
(Polypeptide Sequence of TagGFP)
SEQ ID NO: 12
VSGGEELFAGIVPVLIELDGDVHGHKFSVRGEGEGDADYGKLEIKFICTTGKLPVPWPTLVTTLCYGIQCFARYP
EHMKMNDFFKSAMPEGYIQERTIQFQDDGKYKTRGEVKFEGDTLVNRIELKGKDFKEDGNILGHKLEYSFNSHNV
YIRPDKANNGLEANFKTRHNIEGGGVQLADHYQTNVPLGDGPVLIPINHYLSTQTKISKDRNEARDHMVLLESFS
ACCHTHGMDELYR*
(BoNT/A - UniProt P10845)
SEQ ID NO: 13
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNLFDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
(BoNT/B - UniProt P10844)
SEQ ID NO: 14
MPVTINNFNYNDPIDNNNIIMMEPPFARGTGRYYKAFKITDRIWIIPERYTFGYKPEDFN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVFNNVQENKGASIFNRRGYFSDPALILMHELIHVLHGLY
GIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDFNDINSKLNEGINQAIDNINNFINGCSV
SYLMKKMIPLAVEKLLDFDNTLKKNLLNYIDENKLYLIGSAEYEKSKVNKYLKTIMPFDL
SIYTNDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
(BoNT/C - UniProt P18640)
SEQ ID NO: 15
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTFKLTNNTF
AAQEGFGALSIISISPRFMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKFVELYNELTQIFTE
FNYAKIYNVQNRKIYLSNVYTPVTANILDDNVYDIQNGFNIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVFNTRRNNN
DFNEGYKIIIKRIRGNTNDTRVRGGDILYFDMTINNKAYNLFMKNETMYADNHSTEDIYA
IGLREQTKDINDNIIFQIQPMNNTYYYASQIFKSNFNGENISGICSIGTYRFRLGGDWYR
HNYLVPTVKQGNYASLLESTSTHWGFVPVSE
(BoNT/D - UniProt P19321)
SEQ ID NO: 16
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKFNSLYSDLTNVMSE
VVYSSQYNVKNRTHYFSRHYLPVFANILDDNIYTIRDGFNLTNKGFNIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNFNQAFATAGVAFLLEGFP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYFNSINDSKILSLQNKKNALVDTSGYNAEVRVGDNVQLNTI
YTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDFNIFSKELSNEDINIVYEGQI
LRNVIKDYWGNPLKFDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
(BoNT/E - UniProt Q00496)
SEQ ID NO: 17
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTFSPEYSFRFNDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKFNDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNFNSESA
PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
(BoNT/F - UniProt A7GBG3)
SEQ ID NO: 18
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
(BoNT/G - UniProt Q60393)
SEQ ID NO: 19
MPVNIKXFNYNDPINNDDIIMMEPFNDPGPGTYYKAFRIIDRIWIVPERFTYGFQPDQFN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVFNNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGFNIASKNLKTEFNGQNKAVNKEAY
EEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNFDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDFNDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVFNNYISNISSNAILSLSYRGGRLIDSSGYGATMNVGSDVIFND
IGNGQFKLNNSENSNITAHQSKFVVYDSMFDNFSINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRELNATEVS
SLYWIQSSTNTLKDFWGNPLRYDTQYYLFNQGMONIYIKYFSKASMGETAPRTNFNNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
(Polypeptide Sequence of BoNT/X)
SEQ ID NO: 20
MKLEINKFNYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGFNISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDFSDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNFDLETKKTLDK
FIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
(TeNT - UniProt P04958)
SEQ ID NO: 21
MPITINNFRYSDPVNNDTIIMMEPPYCKGLDIYYKAFKITDRIWIVPERYEFGTKPEDFN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GFNSSVITYPDAQLVPGINGKAIHLVNNESSEVIVHKAMDIEYNDMFNNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND

### EXAMPLES

### Example 1—Generation of a Stable Cell Line

Gene Synthesis and Subcloning

The nucleotide sequences of tagRFPT and tagGFP were derived from Evrogen and synthesized by GeneArt (Thermo Fisher Scientific). The gene product, tRFPT-SNAP25-tGFP, was flanked with attB sequences for Gateway® cloning. The synthesized gene product was then subcloned into a lentivirus vector, pLenti6.3/V5-dest using the BP clonase enzyme kit (Thermo Fisher) according to manufacturer's protocol. The resulting vector, pLenti6.3-tRFPT-SNAP25-tGFP, was transformed in E. coli BL21 cells and selected using Ampicillin antibiotic. Positive bacteria clones were maxi-prepped using Machery-Nagel Endotoxin-free Maxiprep kit according to manufacturer's protocol.

Generation of Lentivirus from HEK293FT Cells

To prepare for generation of lentivirus, HEK293FT cells were cultured in high glucose Dulbecco's modified Eagle's media with 4500 mg/L glucose, supplemented with 10% Fetal Bovine Serum (FBS) (Gibco) then seeded into T75 cm² flask at 80% confluence and incubated overnight at 37° C. with 5% CO2. The cells were then co-transfected with plenti6.3-tRFPT-SNAP25-tGFP plasmid and ViraPower Lentiviral Packaging Mix (Invitrogen Cat No.K497000) using Lipofactamine 3000 reagent (Invitrogen) according to the manual provided by supplier and incubated flask for 6 hours at 37° C. with 5% CO₂. After 6 hours post-transfection, medium that contains lipid-DNA complexes were carefully removed and discarded from the flask, and replaced with 10 ml of pre-warmed medium. The cells were incubated overnight at 37° C. with 5% CO₂. 10 ml of cell supernatant (first batch of virus) was collected after 24 hours post-transfection, and stored in 15 ml conical tubes at 4° C. The collected medium was replaced with 10 ml of pre-warmed medium and the flask was incubated overnight at 37° C. with 5% CO₂. A second batch of virus was collected 48 hours post-transfection. Both batches of supernatant were centrifuged at 2000 rpm for 10 minutes at room temperature to remove cellular debris. The clarified lentiviral supernatant was collected after centrifugation and filtered using a 0.45 μm pore filter to remove any remaining cellular debris. Virus was aliquoted into 1 ml and stored at −80° C.

Measurement of Lentivirus Titre by GFP Selection

HEK293FT cells were seeded in a 96 wells plate (Nunc) at a density of 10000 cells/well in 100 μl of culture medium. Serial dilutions from 10⁻¹ to 10⁻⁴ of virus were made using fresh culture medium with 8 mg/ml (final concentration) Polybrene reagent (Sigma cat no. H9268). Cells were transduced by removing the existing medium and replaced with 100 μl of the prepared dilutions to corresponding well. The plates were incubated overnight at 37° C. with 5% CO2. Culture medium was changed to fresh medium without polybrene the next day. Cells were incubated for additional 3 days before the titer of virus was calculated. The appropriate dilution factor used to calculate the titer in transducing units (TU) per ml based on the percentage of GFP positive cells. The desired transduction range was 1-20%. Hence, titer of virus was determined with the following formula: Titer=(F×C/V)×D, where F=frequency of GFP-positive cells (percent GFP-positive cells/100), C=cell number per well at the time of transduction, V=volume of inoculum in ml (0.1 ml) and D=lentivirus dilution factor.

Generation of ReNcell VM Stable Cell Line from Lentivirus

ReNcell VM (Millipore) cells were seeded in 24 wells coated with laminin (final concentration 20 μg/ml) at 80% confluence and incubated overnight at 37° C. with 5% CO₂. Medium was removed and 500 μl of lentivirus added per well with Polybrene reagent at a final concentration of 8 mg/ml. Cells were incubated overnight at 37° C. with 5% CO₂. Medium was replaced with fresh medium without polybrene the next day. Transduced cells were expanded and FAC-sorted using the GFP wavelength.

Results

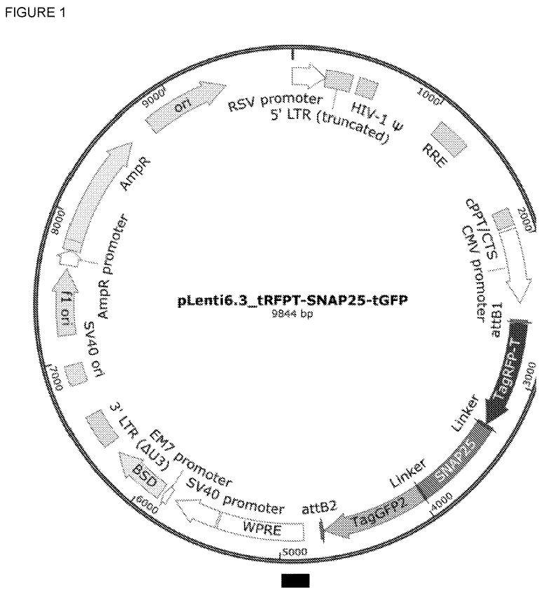

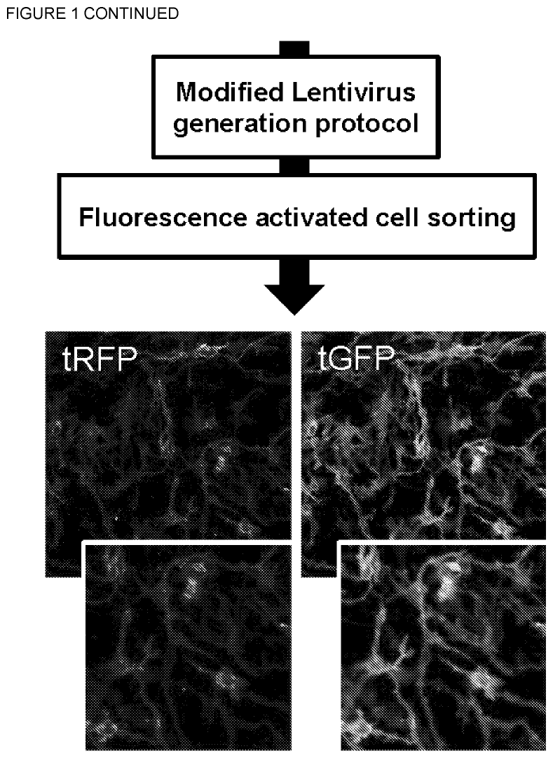

The assay construct consisting of full-length SNAP25 flanked by tagRFPT and tagGFP was cloned into a lentivirus vector backbone. Generation of stable cell line was achieved using a modified lentivirus generation protocol consisting of lipofection of construct with lentiviral packaging plasmids into the HEK293T cell line. The resulting lentivirus was purified and added onto ReNcell VM cells, which were eventually sorted using FACS (see FIG. 1), and the generation of the stable cell line confirmed. This v-myc immortalised cell line is derived from human neural progenitor cells (NPCs), which is genetically closer to native human neurons as compared to cancer cell lines, and is therefore a better neuronal cell model for use in the assays described herein.

### Example 2—the Construct is Sensitive to BoNT/A Cleavage Materials and Methods

Perkin Elmer CellCarrier 384 Ultra™ imaging plates and Nunc 24-well tissue culture dishes were incubated with 20 μg/mL laminin (Invitrogen) overnight at 4° C.

Imaging

The stable cell line of the invention (referred to as the ReD SNAPR cell line) was differentiated according to the ReNcell VM cell manufacturer's protocol. In brief, cells were seeded on pre-coated Perkin Elmer CellCarrier 384 Ultra™ imaging plates at 3000 cells per well. Cells were maintained in ReNcell NSC Maintenance Media without growth factors (EGF & FGF2) (differentiation media) for 14 days, with media change every 3 days. Cells were incubated with 100 nM BoNT/A in differentiation media for 48 hours. Cells were fixed with fixative (4% paraformaldehyde and 2% sucrose). Fixed cells were imaged using Opera™ Phenix.

Western Blot

ReD SNAPR cells were differentiated according to the ReNcell VM cell manufacturer's protocol. Briefly, cells were seeded on Nunc 24-well tissue culture dish at 30,000 cells per well. Cells were maintained in ReNcell NSC Maintenance Media without growth factors (EGF & FGF2) (differentiation media) for 14 days, with media change every 3 days. Cells were incubated with 100 nM BoNT/A in differentiation media for 48 hours. The medium was aspirated and cells were lysed with NP-40 lysis buffer (150 mM NaCl, 1% NP-40, 50 nM Tris-Cl, pH 8.0). To prepare samples for loading into SDS-PAGE gel, 10% DTT and 6X loading buffer (BioRad) was added to the samples and boiled for 5 mins. 20 μL of samples were added into each lane of a NuPAGE Bis-tris 4-12% gel (Thermo Fisher) and run at 120V until the dye front ran out. The gel was transferred onto a nitrocellulose membrane and probed with anti-tRFP and anti-tag (CGY)FP (Evrogen) overnight.

Results

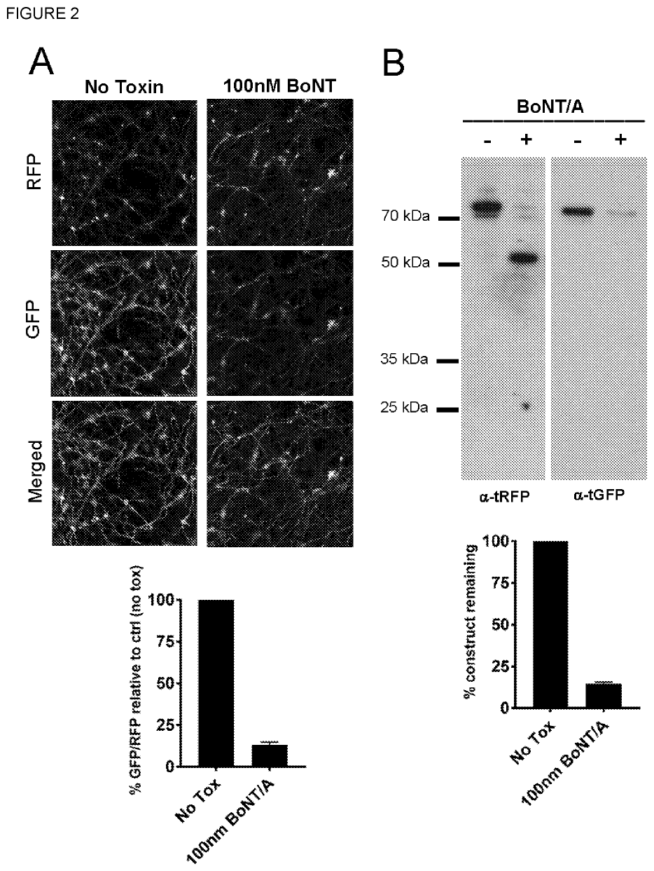

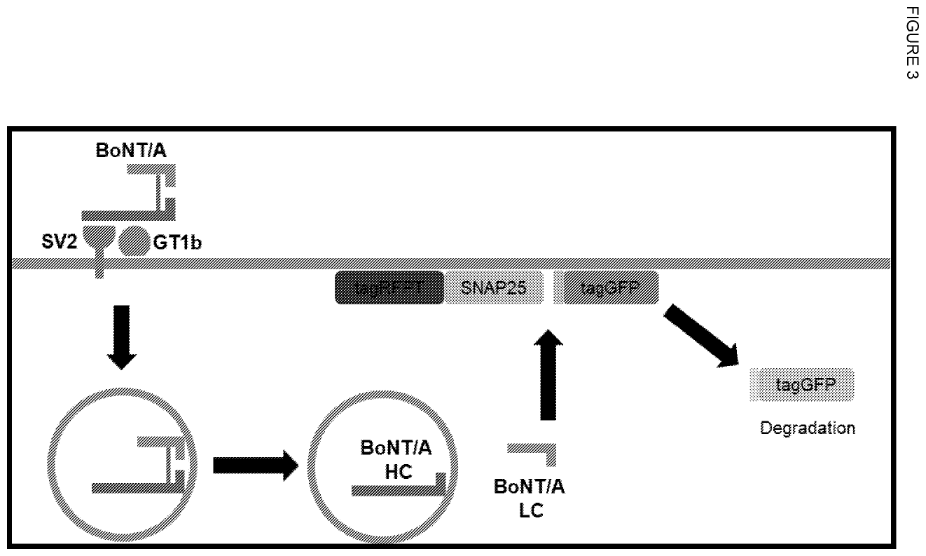

FIG. 2 demonstrates that the construct of the assay is sensitive to degradation by BoNT/A. Conventional cell-based assays focus on FRET interactions or direct western blot detection of cleaved SNAP25 in the cell lysate, which are not suitable for high-throughput screening (HTS) applications. In normal circumstances, the BoNT/A-cleaved C-terminal fragment of full-length SNAP25 is hardly detectable due to the small molecular weight, hence its fate is usually unknown. This previously-unrecognized observation shows that the C-terminal fragment is degraded along with the fluorophore that is attached with it. The relative ease of this methodology is highly suitable for a range low to high-throughput applications.

FIG. 3 presents a schematic showing BoNT/A-mediated degradation of the C-terminus of the SNAP25 construct. After internalisation of BoNT/A in ReD SNAPR cells, the light chain of BoNT/A enters the cytoplasm and cleaves tagRFPT-SNAP25-tagGFP (stably expressed in ReD SNAPR cells). This results in the degradation of the C-terminal fragment, while retaining the N-terminal construct. The degradation can be detected using fluorescence microscopy and Western blot.

### Example 3—Improving Sensitivity of the ReD SNAPR Cells to BoNT/A

ReD SNAPR cells were seeded onto 384 well plates as described above. For enhanced differentiation, ReD SNAPR cells were cultured in normal ReNcell media with 10 ng/mL GDNF and 1 mM d-cAMP (cell permeable cAMP). Various concentrations (0-1 μM) of BoNT/A was added to normal and ReDS media where ReDS media contained 10 ng/mL GDNF, 1 mM d-cAMP, 2 mM CaCl₂ and 56 mM KCl. Differentiated ReD SNAPR cells were intoxicated with BoNT/A-containing medium, fixed and imaged as described above.

Results

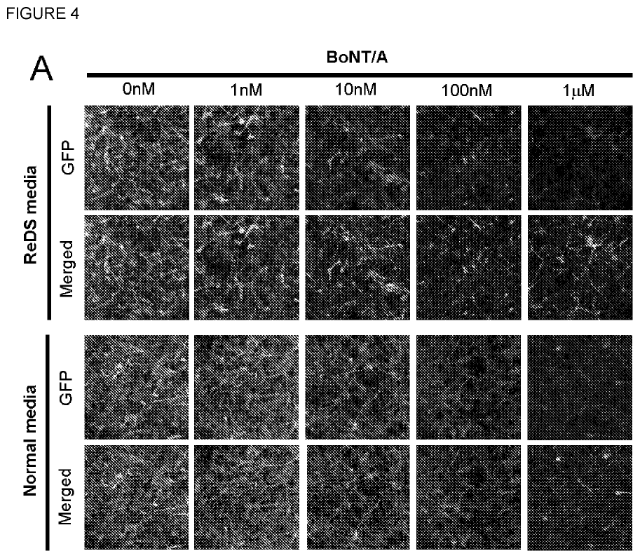

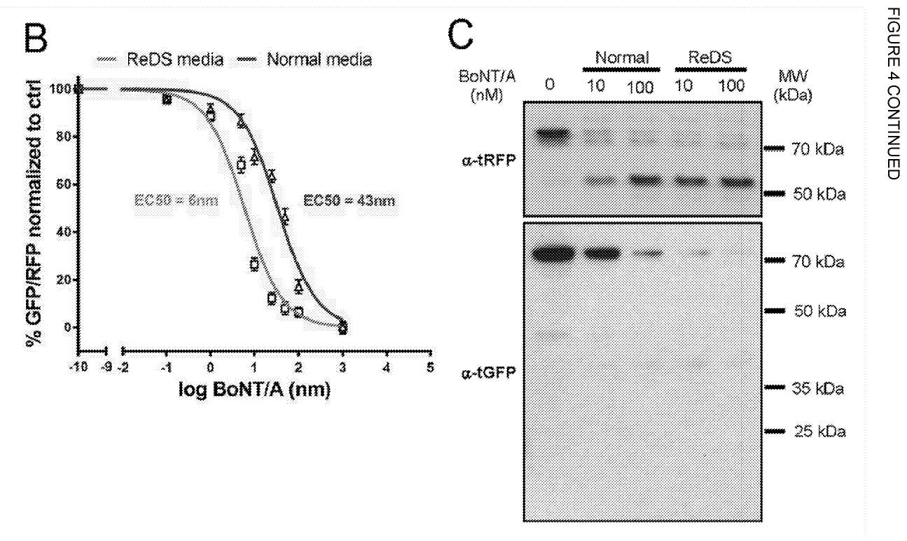

FIG. 4 shows that addition of GDNF and d-cAMP during differentiation, and high potassium conditions during intoxication improved sensitivity of the cell line to BoNT/A. The dose response curves and EC₅₀ values of BoNT/A in the assay were determined (see FIG. 4B) with low nM values when the cells were exposed to ReDS medium.

### Example 4—Thioredoxin Reductase (TrxR1) as an Assay Control

ReD SNAPR cells were seeded onto 384 well plates and differentiated as described above. Differentiated cells were treated with 25 nmol of either a siRNA non-targeting control, NT3 or siRNA against TrxR1 using Lipofectamine RNAimax according to the manufacturer's protocol and left on cells for 72 hours. ReDS medium containing 10 nM BoNT/A was added to cells for 48 hours and then fixed and imaged as described above. Briefly, cells were fixed and an antibody against TrxR1 was used to detect TrxR1 and fluorescence imaged using Opera Phenix. Mean fluorescence intensity levels of GFP, RFP and Far-red channels were captured and measured.

Results

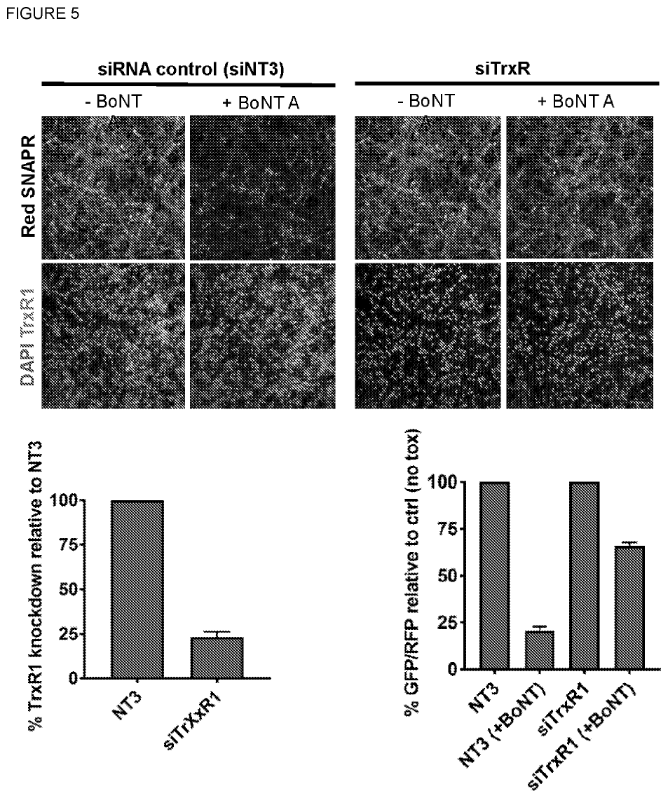

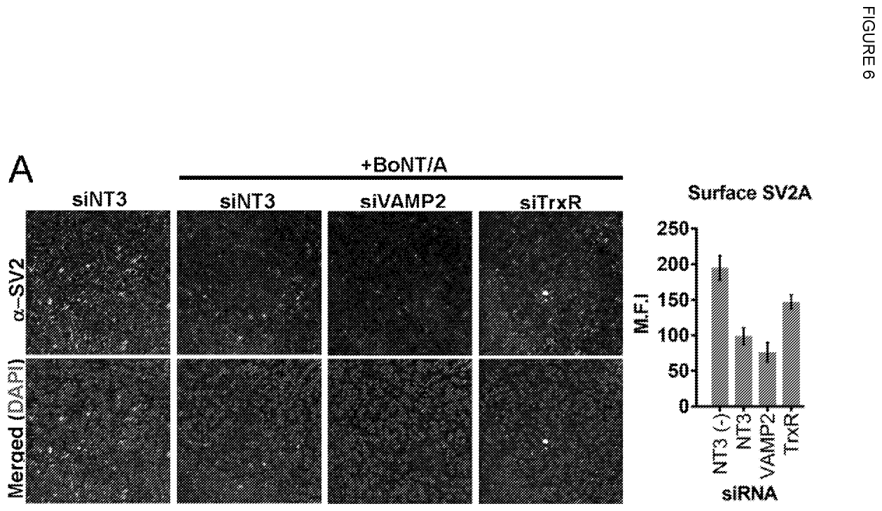

FIG. 5 shows that siTrxR-treated cells are more resistant to BoNT/A-mediated cleavage as compared to control. Thus, TrxR1 can be used as a suitable positive control to identify genes involved in the intracellular trafficking of BoNT/A. In particular TrXR1 knock-down can be used to show that BoNT/A intoxication can be rescued, thus further validating genes identified in an assay.

### Example 5—Use of BoNT Receptor SV2

ReNcell VM cells were seeded onto 384 well plates and differentiated as previously described. Differentiated cells were treated with 25 nmol of either siNT3, siVAMP2 or siTrxR using Lipofectamine RNAimax according to manufacturer's protocol and left on cells for 72 hours. ReDS medium containing 10 nM BoNT/A was added to cells for 48 hours and then fixed. For immunostaining, the cells were blocked with 0.5% BSA/PBS for 1 hour and an antibody against SV2A (Cell Signaling, #66724) was added to cells and incubated for at least 1 hour. An Alexa-488 conjugated secondary antibody was added into cells for 1 hour and cells were imaged using Opera Phenix. Cells were then imaged using Opera Phenix with GFP and DAPI channels shown.

Results

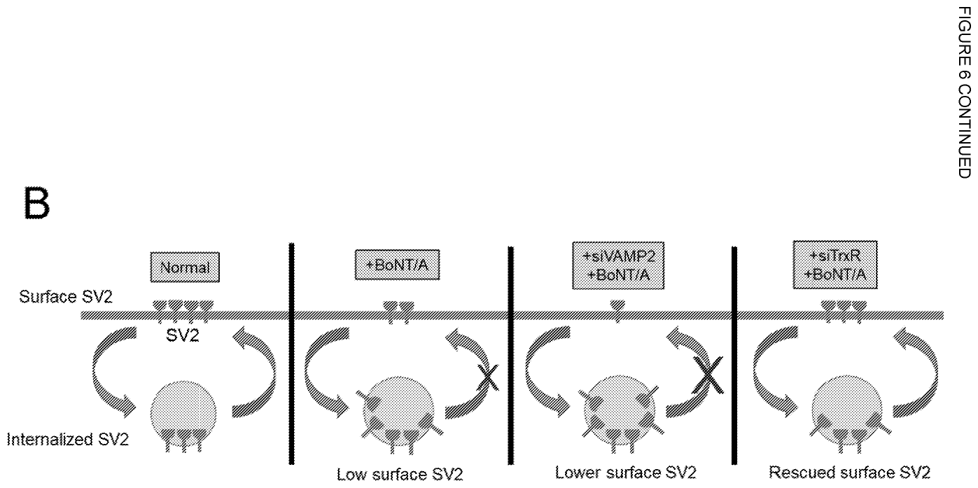

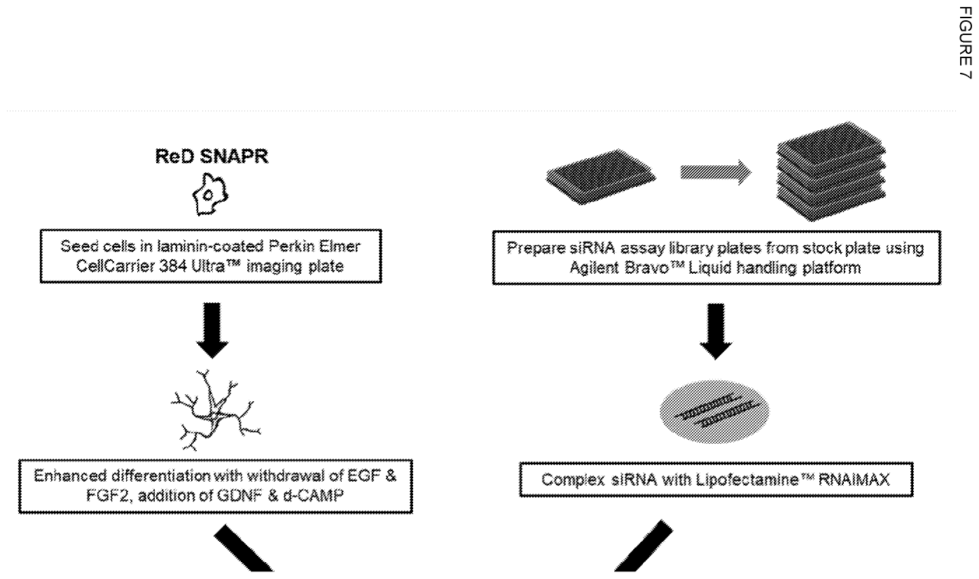

Although SV2 is the major receptor for BoNT/A, it has not previously been further studied for its post-intoxication itinerary in the cell. FIG. 6 shows that BoNT/A intoxication leads to decreased SV2 at the cell surface which could be the typical consequence of BoNT/A-mediated defective trafficking to the cell surface. In this case, the recycling of SV2 back to the cell surface is blocked. Hence SV2 can be used as a readout for BoNT/A intoxication.

Many genes can regulate BoNT/A activity in the cell. An example of indirect regulation would be at the level of BoNT receptor SV2 trafficking (instead of modulation of toxin activity itself). To sieve out candidates involved in SV2 trafficking, the surface SV2 staining may be an ideal selection criteria. An example shown here are cells depleted with VAMP2, which upon BoNT/A intoxication resulted in lower surface SV2 staining. This could be due to the synergistic action of blocking vesicle exocytosis at the cell surface via decreased VAMP2 and BoNT/A intoxication.

Surface SV2 can be rescued via depleting TrxR, which shows that TrxR itself does not affect exocytosis of SV2 at the cell surface but directly modulating BoNT/A activity via release of its light chain (LC). This inadvertently results in the restoration of surface SV2 due to decreased BoNT/A LC in the cytoplasm.

Thus, SV2 is useful in an assay of the invention as it can be used to sieve out gene candidates directly involved in BoNT/A trafficking from those that modulate the trafficking of the BoNT/A receptor SV2.

### Example 6—Genome-Wide siRNA Screen

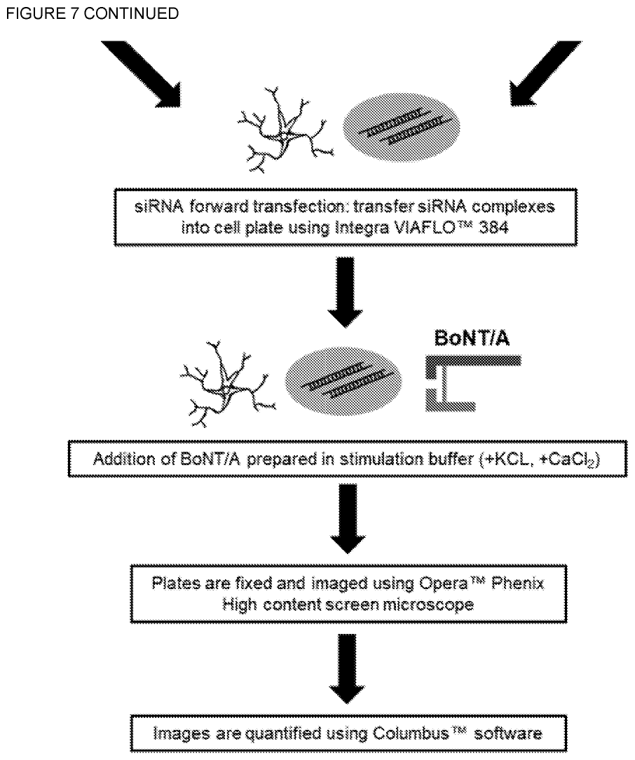

FIG. 7 provides a diagram showing a method for carrying out a genome-wide siRNA screen with a cell line of the invention. The ReD SNAPR cells are plated and differentiated as described above. An siRNA library is prepared and complexed with Lipofectamine™ RNAiMAX using standard protocols, and the ReD SNAPR cells are transfected with the siRNA. BoNT/A in stimulation buffer is added to the cells prior to fixing and imaging using Opera™ Phenix and quantification with Columbus™ software.

Positive hits may be subjected to further validation by assessing recovery in the presence of siRNA against TrxR1. Confirmation that the genes directly regulate BoNT activity are confirmed by way of SV2 cell surface staining as described above.

### Example 7—Identifying Prophylactic Anti-Botulism Therapeutics

The ReD SNAPR cells are plated and differentiated as described above and exposed to an agent (e.g. a small-molecule drug). BoNT/A in stimulation buffer is added to the cells prior to fixing and imaging using Opera™ Phenix and quantification with Columbus™ software.

An agent is identified as a prophylactic anti-botulism therapeutic if cleavage of the construct is inhibited.

### Example 8—Identifying Post-Intoxication Anti-Botulism Therapeutics

The ReD SNAPR cells are plated and differentiated as described above and BoNT/A in stimulation buffer is added to the cells and cleavage of the construct (loss of GFP) is observed. Next, the cells are exposed to an agent (e.g. a small-molecule drug). Finally, cells are fixed and imaged using Opera™ Phenix and quantification with Columbus™ software.

An agent is identified as a post-intoxication anti-botulism therapeutic if recovery of GFP is observed.

### Example 9—Identifying BoNT Sensitising Agents

The ReD SNAPR cells are plated and differentiated as described above and exposed to an agent (e.g. a small-molecule drug). BoNT/A in stimulation buffer is added to the cells prior to fixing and imaging using Opera™ Phenix and quantification with Columbus™ software.

An agent is identified as a BoNT sensitising agent if cleavage of the construct is improved (e.g. occurs faster or more cleavage is evident). The sensitising agent is taken forward for further study for use as a companion product to modulate local activity of clostridial neurotoxins (e.g. to allow reduced dosage and minimise spread to other tissues).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for identifying a gene that regulates clostridial neurotoxin activity, the method comprising:
a. providing a sample of human neuronal cells expressing a polypeptide that comprises a C-terminal detectable label, wherein the polypeptide is cleavable by a clostridial neurotoxin;
b. altering expression of a target gene of the cells;
c. contacting the cells with the clostridial neurotoxin;
d. measuring an amount of C-terminal detectable label, thereby quantifying clostridial neurotoxin activity; and
e. identifying the target gene as a regulator of clostridial neurotoxin activity when the quantified clostridial neurotoxin activity is different to the quantified clostridial neurotoxin activity when expression of the target gene is unaltered; or
f. identifying that the target gene is not a regulator of clostridial neurotoxin activity when the quantified clostridial neurotoxin activity is equivalent to the quantified clostridial neurotoxin activity when expression of the target gene is unaltered.

2. The method according to claim 1, wherein expression is altered by downregulating expression of the target gene.

3. The method according to claim 2, wherein the target gene is identified as a positive regulator of clostridial neurotoxin activity when the quantified clostridial neurotoxin activity is less than the quantified clostridial neurotoxin activity when expression of the target gene is unaltered.

4. The method according to claim 2 or 3, wherein the target gene is identified as a negative regulator of clostridial neurotoxin activity when the quantified clostridial neurotoxin activity is greater than the quantified clostridial neurotoxin activity when expression of the target gene is unaltered.

5. The method according to any one of the preceding claims, wherein a plurality of samples of human neuronal cells are provided, and wherein the expression of a different target gene is altered in each sample of human neuronal cells.

6. The method according to claim 5, wherein the expression of a different target gene is altered in each sample using a human RNAi library.

7. The method according to any one of the preceding claims, further comprising determining whether the target gene is a direct regulator of clostridial neurotoxin activity or an indirect regulator of clostridial neurotoxin activity.

8. The method according to claim 7, wherein the direct regulator regulates clostridial neurotoxin activity at the level of:
i. binding of the clostridial neurotoxin to the cell;
ii. internalisation of the clostridial neurotoxin;
iii. translocation of the clostridial neurotoxin L-chain out of the endosome;
iv. catalysis; and/or
v. persistence of the L-chain activity within the cell cytoplasm.

9. The method according to claim 7 or 8, wherein the indirect regulator regulates cellular trafficking of a clostridial neurotoxin receptor.

10. The method according to any one of claims 7-9, wherein the determining comprises detecting the presence or absence of a clostridial neurotoxin receptor of the cell when expression of the target gene has been altered.

11. The method according to any one of claims 7-10, wherein:
detecting a decreased amount of a clostridial neurotoxin receptor on the surface of the cell when expression of the target gene has been altered (preferably downregulated) when compared to an equivalent cell contacted with clostridial neurotoxin in which the expression of the target gene is unaltered indicates that the target gene indirectly regulates clostridial neurotoxin activity; or
detecting an equivalent or greater (preferably greater) amount of a clostridial neurotoxin receptor on the surface of the cell when expression of the target gene has been altered (preferably downregulated) when compared to an equivalent cell contacted with clostridial neurotoxin in which the expression of the target gene is unaltered indicates that the target gene directly regulates clostridial neurotoxin activity.

12. The method according to any one of claims 9-11, wherein the clostridial neurotoxin receptor is Synaptic Vesicle Glycoprotein 2A (SV2).

13. A method for identifying an agent that regulates clostridial neurotoxin activity, the method comprising:
a. providing a sample of human neuronal cells expressing a polypeptide that comprises a C-terminal detectable label, wherein the polypeptide is cleavable by a clostridial neurotoxin;
b. contacting the cells with the clostridial neurotoxin and an agent, wherein the contacting is sequential or simultaneous;
c. measuring an amount of C-terminal detectable label, thereby quantifying clostridial neurotoxin activity; and
d. identifying the agent as a regulator of clostridial neurotoxin activity when the quantified clostridial neurotoxin activity is different to the quantified clostridial neurotoxin activity in the absence of the agent; or
e. identifying that the agent is not a regulator of clostridial neurotoxin activity when the quantified clostridial neurotoxin activity is equivalent to the quantified clostridial neurotoxin activity in the absence of the agent.

14. The method according to claim 13, wherein the agent is identified as a negative regulator of clostridial neurotoxin activity when the quantified clostridial neurotoxin activity is less than the quantified clostridial neurotoxin activity in the absence of the agent.

15. The method according to claim 13 or 14, wherein the agent is identified as a positive regulator of clostridial neurotoxin activity when the quantified clostridial neurotoxin activity is greater than the quantified clostridial neurotoxin activity in the absence of the agent.

16. The method according to any one of claims 13-15, further comprising determining whether the agent is a direct regulator of clostridial neurotoxin activity or an indirect regulator of clostridial neurotoxin activity.

17. The method according to claim 16, wherein the direct regulator regulates clostridial neurotoxin activity at the level of:
i. binding of the clostridial neurotoxin to the cell;
ii. internalisation of the clostridial neurotoxin;
iii. translocation of the clostridial neurotoxin L-chain out of the endosome;
iv. catalysis; and/or
v. persistence of the L-chain activity within the cell cytoplasm.

18. The method according to claim 16 or 17 wherein the indirect regulator regulates cellular trafficking of a clostridial neurotoxin receptor.

19. The method according to any one of claims 16-18, wherein the determining comprises detecting the presence or absence of a clostridial neurotoxin receptor of the cell when the cell has been targeted with the agent.

20. The method according to any one of claims 16-19, wherein:
detecting a decreased amount of a clostridial neurotoxin receptor on the surface of the cell when the cell has been contacted with the agent when compared to an equivalent cell contacted with clostridial neurotoxin that has not been contacted with the agent indicates that the agent indirectly regulates clostridial neurotoxin activity; or
detecting an equivalent or greater (preferably greater) amount of a clostridial neurotoxin receptor on the surface of the cell when the cell has been contacted with the agent when compared to an equivalent cell contacted with clostridial neurotoxin that has not been contacted with the agent indicates that the agent directly regulates clostridial neurotoxin activity.

21. The method according to any one of claims 18-20, wherein the clostridial neurotoxin receptor is Synaptic Vesicle Glycoprotein 2A (SV2).

22. A human neuronal cell expressing a polypeptide, wherein the polypeptide is cleavable by a clostridial neurotoxin and comprises a C-terminal detectable label.

23. The method according to any one of claims 1-21 or the cell according to claim 22, wherein the human neuronal cell is a non-cancer cell.

24. The method or cell according to any one of the preceding claims, wherein the human neuronal cell is an immortalized human neural progenitor cell, or preferably wherein the human neuronal cell has been derived (e.g. differentiated) from an immortalized human neural progenitor cell.

25. The method or cell according to any one of the preceding claims, wherein the polypeptide further comprises an N-terminal detectable label, and wherein the N-terminal detectable label is different to the C-terminal detectable label.

26. The method or cell according to claim 25, wherein one of the detectable labels is red fluorescent protein (RFP) and one of the detectable labels is selected from green fluorescent protein (GFP), cyan fluorescent protein (CFP), and yellow fluorescent protein (YFP).

27. The method or cell according to any one of the preceding claims, wherein the polypeptide comprises an N-terminal RFP and a C-terminal GFP.

28. The method or cell according to any one of the preceding claims, wherein the polypeptide:
a. is encoded by a nucleotide sequence comprising:
i. a nucleotide sequence having at least 70% sequence identity to SEQ ID NO: 3;
ii. a nucleotide sequence having at least 70% sequence identity to SEQ ID NO: 9; and/or
iii. a nucleotide sequence having at least 70% sequence identity to SEQ ID NO: 11; or
b. is encoded by a nucleotide sequence having at least 70% sequence identity to SEQ ID NO: 1; or
c. comprises a polypeptide sequence comprising:
i. a polypeptide sequence having at least 70% sequence identity to SEQ ID NO: 4;
ii. a polypeptide sequence having at least 70% sequence identity to SEQ ID NO: 10; and/or
iii. a polypeptide sequence having at least 70% sequence identity to SEQ ID NO: 12; or
d. comprises a polypeptide sequence having at least 70% sequence identity to SEQ ID NO: 2.

29. The method or cell according to any one of the preceding claims, wherein the polypeptide:
a. is encoded by a nucleotide sequence comprising:
i. a nucleotide sequence having at least 80% sequence identity to SEQ ID NO: 3;
ii. a nucleotide sequence having at least 80% sequence identity to SEQ ID NO: 9; and/or
iii. a nucleotide sequence having at least 80% sequence identity to SEQ ID NO: 11; or
b. is encoded by a nucleotide sequence having at least 80% sequence identity to SEQ ID NO: 1; or
c. comprises a polypeptide sequence comprising:
i. a polypeptide sequence having at least 80% sequence identity to SEQ ID NO: 4;
ii. a polypeptide sequence having at least 80% sequence identity to SEQ ID NO: 10; and/or
iii. a polypeptide sequence having at least 80% sequence identity to SEQ ID NO: 12; or
d. comprises a polypeptide sequence having at least 80% sequence identity to SEQ ID NO: 2.

30. The method or cell according to any one of the preceding claims, wherein the polypeptide:
a. is encoded by a nucleotide sequence comprising:
i. a nucleotide sequence having at least 90% sequence identity to SEQ ID NO: 3;
ii. a nucleotide sequence having at least 90% sequence identity to SEQ ID NO: 9; and/or
iii. a nucleotide sequence having at least 90% sequence identity to SEQ ID NO: 11; or
b. is encoded by a nucleotide sequence having at least 90% sequence identity to SEQ ID NO: 1; or
c. comprises a polypeptide sequence comprising:
i. a polypeptide sequence having at least 90% sequence identity to SEQ ID NO: 4;
ii. a polypeptide sequence having at least 90% sequence identity to SEQ ID NO: 10; and/or
iii. a polypeptide sequence having at least 90% sequence identity to SEQ ID NO: 12; or
d. comprises a polypeptide sequence having at least 90% sequence identity to SEQ ID NO: 2.

31. The method or cell according to any one of the preceding claims, wherein the polypeptide:
a. is encoded by a nucleotide sequence comprising:
i. SEQ ID NO: 3;
ii. SEQ ID NO: 9; and
iii. SEQ ID NO: 11; or
b. is encoded by a nucleotide sequence comprising SEQ ID NO: 1; or
c. comprises:
i. SEQ ID NO: 4;
ii. SEQ ID NO: 10; and
iii. SEQ ID NO: 12; or
d. comprises SEQ ID NO: 2.

32. A nucleotide sequence encoding a polypeptide, wherein the polypeptide is cleavable by a clostridial neurotoxin and comprises an N-terminal RFP and a C-terminal GFP, wherein the nucleotide sequence comprises:
a. a nucleotide sequence having at least 70% sequence identity to SEQ ID NO: 3;
b. a nucleotide sequence having at least 70% sequence identity to SEQ ID NO: 9; and
c. a nucleotide sequence having at least 70% sequence identity to SEQ ID NO: 11.

33. The nucleotide sequence according to claim 32, wherein the nucleotide sequence has at least 70% sequence identity to SEQ ID NO: 1.

34. The nucleotide sequence according to claim 32 or 33, wherein the nucleotide sequence has at least 80% sequence identity to SEQ ID NO: 1.

35. The nucleotide sequence according to any one of claims 32-34, wherein the nucleotide sequence has at least 90% sequence identity to SEQ ID NO: 1.

36. The nucleotide sequence according to any one of claims 32-35, wherein the nucleotide sequence comprises SEQ ID NO: 1.

37. The method or cell according to any one of claims 1-31 or the nucleotide sequence according to any one of claims 32-36, wherein the nucleotide sequence consists of SEQ ID NO: 1.

38. A vector comprising the nucleotide sequence according to any one of claims 32-37.

39. A polypeptide that is cleavable by a clostridial neurotoxin and comprises an N-terminal RFP and a C-terminal GFP, wherein the polypeptide comprises:
a. a polypeptide sequence having at least 70% sequence identity to SEQ ID NO: 4;
b. a polypeptide sequence having at least 70% sequence identity to SEQ ID NO: 10; and
c. a polypeptide sequence having at least 70% sequence identity to SEQ ID NO: 12.

40. The polypeptide according to claim 39, wherein the polypeptide sequence has at least 70% sequence identity to SEQ ID NO: 2.

41. The polypeptide according to claim 39 or 40, wherein the polypeptide sequence has at least 80% sequence identity to SEQ ID NO: 2.

42. The polypeptide according to any one of claims 39-41, wherein the polypeptide sequence has at least 90% sequence identity to SEQ ID NO: 2.

43. The polypeptide according to any one of claims 39-42, wherein the polypeptide sequence comprises SEQ ID NO: 2.

44. The method or cell according to any one of claim 1-31 or 37 or the polypeptide according to any one of claims 39-43, wherein the polypeptide sequence consists of SEQ ID NO: 2.

45. The method according to any one of claim 1-31, 37 or 44, wherein the method comprises contacting the cells with a buffer comprising glial cell-derived neurotrophic factor (GDNF), cell-permeable cyclic adenosine monophosphate (cAMP), CaCl₂ and KCl.

46. The method according to claim 45, wherein the GDNF is present in the buffer at a concentration of 1-100 ng/ml, cAMP is present in the buffer at a concentration of 0.1-5 mM, CaCl₂ is present in the buffer at a concentration of 0.1-7 mM and/or KCl is present in the buffer at a concentration of 1-100 mM.

47. A kit comprising:
a. the cell according to any one of claim 22-31, 37 or 44; or
b. the nucleotide sequence according to any one of claims 32-37; or
c. the vector according to claim 38; and
d. optionally instructions for use of the same.

48. The kit according to claim 47, further comprising a buffer, said buffer comprising GDNF, cell-permeable cyclic adenosine monophosphate (cAMP), CaCl₂ and KCl.

49. A composition comprising:
a. a clostridial neurotoxin; and
b. a buffer, the buffer comprising GDNF, cell-permeable cyclic adenosine monophosphate (cAMP), CaCl₂ and KCl.

50. The kit according to claim 48 or the composition according to claim 49, wherein the GDNF is present in the buffer at a concentration of 1-100 ng/ml, cAMP is present in the buffer at a concentration of 0.1-5 mM, CaCl₂ is present in the buffer at a concentration of 0.1-7 mM and KCl is present in the buffer at a concentration of 1-100 mM.


SEQUENCE LISTING (26 sequences)

SEQ ID NO: 1 - DNA, 2109 bp, Artificial Sequence
  Construct TagRFPT-SNAP25-TagGFP
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaagggctcgggctcgggctcgggcgtggccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactcgatcgtgtcgaagaaggcatgaaccatatcaaccaagacatgaaggaggctgagaaaaatttaaaagatttagggaaatgctgtggccttttcatatgtccttgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttacggcggctcgggctcgggcgtgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaa

SEQ ID NO: 2 - protein, 702 aa, Artificial Sequence
  Construct TagRFPT-SNAP25-TagGFP
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYKGSGSGSGVAEDADMRNELEEMQRRADQLADESLESTRRMLQLVEESKDAGIRTLVMLDEQGEQLDRVEEGMNHINQDMKEAEKNLKDLGKCCGLFICPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGYGGSGSGVSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYR

SEQ ID NO: 3 - DNA, 732 bp, Artificial Sequence
  TagRFPT
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaaccgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaatggcatggacgagctgtacaag

SEQ ID NO: 4 - protein, 244 aa, Artificial Sequence
  TagRFPT
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPGVYYVDHRLERIKEADKETYVEQHEVAVARYCDLPSKLGHKLNGMDELYK

SEQ ID NO: 5 - DNA, 21 bp, Artificial Sequence
  Glycine-Serine rich linker 1
ggctcgggctcgggctcgggc

SEQ ID NO: 6 - protein, 7 aa, Artificial Sequence
  Glycine-Serine rich linker 1
GSGSGSG

SEQ ID NO: 7 - DNA, 18 bp, Artificial Sequence
  Glycine-Serine rich linker 2
ggcggctcgggctcgggc

SEQ ID NO: 8 - protein, 6 aa, Artificial Sequence
  Glycine-Serine rich linker 2
GGSGSG

SEQ ID NO: 9 - DNA, 621 bp, Artificial Sequence
  SNAP25
gtggccgaagacgcagacatgcgcaatgagctggaggagatgcagcgaagggctgaccagttggctgatgagtcgctggaaagcacccgtcgtatgctgcaactggttgaagagagtaaagatgctggtatcaggactttggttatgttggatgaacaaggagaacaactcgatcgtgtcgaagaaggcatgaaccatatcaaccaagacatgaaggaggctgagaaaaatttaaaagatttagggaaatgctgtggccttttcatatgtccttgtaacaagcttaaatcaagtgatgcttacaaaaaagcctggggcaataatcaggacggagtggtggccagccagcctgctcgtgtagtggacgaacgggagcagatggccatcagtggcggcttcatccgcagggtaacaaatgatgcccgagaaaatgaaatggatgaaaacctagagcaggtgagcggcatcatcgggaacctccgtcacatggccctggatatgggcaatgagatcgatacacagaatcgccagatcgacaggatcatggagaaggctgattccaacaaaaccagaattgatgaggccaaccaacgtgcaacaaagatgctgggaagtggttac

SEQ ID NO: 10 - protein, 207 aa, Artificial Sequence
  SNAP25
VAEDADMRNELEEMQRRADQLADESLESTRRMLQLVEESKDAGIRTLVMLDEQGEQLDRVEEGMNHINQDMKEAEKNLKDLGKCCGLFICPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGY

SEQ ID NO: 11 - DNA, 717 bp, Artificial Sequence
  TagGFP
gtgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaa

SEQ ID NO: 12 - protein, 238 aa, Artificial Sequence
  TagGFP
VSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYR

SEQ ID NO: 13 - protein, 1296 aa, Clostridium botulinum [sequence omitted]

SEQ ID NO: 14 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 15 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 16 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 17 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 18 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 19 - protein, 1297 aa, Clostridium botulinum
  SITE (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 20 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 21 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 22 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XDXXXLL

SEQ ID NO: 23 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLL

SEQ ID NO: 24 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXIL

SEQ ID NO: 25 - protein, 7 aa, Artificial Sequence
  Leucine-Based Motif
  SITE (1)..(1): Xaa can be any naturally occurring amino acid
  SITE (3)..(5): Xaa can be any naturally occurring amino acid
XEXXXLM

SEQ ID NO: 26 - protein, 4 aa, Artificial Sequence
  Tyrosine-Based Motif
  SITE (2)..(3): Xaa can be any naturally occurring amino acid
  VARIANT (4)..(4): Xaa is a hydrophobic amino acid
YXXX